(19) United States
(12) Patent Application Publication
(10) Pub. No.: US 2025/0109436 A1
(43) Pub. Date: Apr. 3, 2025

(54) METHODS, COMPOSITIONS, AND SYSTEMS FOR LONG READ SINGLE MOLECULE SEQUENCING

(71) Applicant: Reticula, Inc., San Diego, CA (US)

(72) Inventor: Nava Edmond WHITEFORD, Chiba (JP)

(73) Assignee: Reticula, Inc., San Diego, CA (US)

(21) Appl. No.: 18/836,649

(22) PCT Filed: Feb. 7, 2023
(86) PCT No.: PCT/US2023/062148

§ 371 Date: Aug. 7, 2024

Related U.S. Application Data
(60) Provisional application No. 63/308,016, filed on Feb. 8, 2022.
(60) Provisional application No. 63/312,059, filed on Feb. 20, 2022.
(60) Provisional application No. 63/312,060, filed on Feb. 20, 2022.

(51) Int. Cl. C12Q 1/6874

(52) U.S. Cl. CPC C12Q 1/6874

(57) ABSTRACT

The present disclosure in some aspects relates to real-time nucleic acid sequencing comprising i) cluster decimation by deactivating some strands in a cluster, ii) bringing the non-deactivated strands in the cluster out of sync with each other during sequencing, and/or iii) one or more detectable label deactivation steps, such as stochastic photobleaching that occurs during sequential nucleotide incorporation in a sequencing-by-synthesis reaction. Deactivation of the detectable label(s) of a particular labeled nucleotide may occur prior to, during, and/or after the incorporation of the next nucleotide, for instance, a labeled nucleotide that forms a phosphodiester bond with the particular labeled nucleotide. Also described herein are methods of analyzing sequencing data obtained from the sequencing methods.

### CROSS-REFERENCE TO RELATED APPLICATIONS

This application claims priority to U.S. Provisional Patent Application No. 63/308,016, filed Feb. 8, 2022, entitled “Long read single molecule colony sequencing,” U.S. Provisional Patent Application No. 63/312,059, filed Feb. 20, 2022, entitled “Long read single molecule colony sequencing,” and U.S. Provisional Patent Application No. 63/312,060, filed Feb. 20, 2022, entitled “Optical approach for TIRF imaging systems with particular reference to DNA/RNA sequencing,” which applications are herein incorporated by reference in their entireties for all purposes.

### FIELD

The present disclosure generally relates to methods and compositions for determining a sequence of a nucleic acid molecule, including methods and compositions for single-molecule sequencing and/or real-time sequencing of a plurality of nucleic acid molecules.

### BACKGROUND

The analysis of nucleic acid molecules is an extremely complex endeavor which typically requires accurate, rapid characterization of large numbers of nucleic acid molecules via high throughput DNA sequencing. The determination of nucleic acid sequences remains a laborious and difficult task, particularly in comparison to cheaper probe based methods such as qPCR (also called real-time PCR). Simplifying and reducing the cost of sequencing therefore remains an important problem. The present disclosure addresses these and other needs.

### BRIEF SUMMARY

Known nucleic acid sequencing-by-synthesis (SBS) methods are cyclic and require deactivation of signal from a labeled nucleotide incorporated in one cycle and removal of labeled nucleotides that are not incorporated in that cycle, prior to introducing labeled nucleotides for the next cycle. For example, in some existing methods, dye-labeled “A” nucleotides (e.g., dATP labeled with a first fluorophore) would be introduced into a flow cell, incorporated and detected at particular spots in the flow cell (e.g., indicating a base “T” in the template molecules at those spots), and then the dye in the incorporated nucleotides at those particular spots would be bleached (and unincorporated dye-labeled nucleotides removed from the flow cell) before dye-labeled “T” nucleotides (e.g., dTTP labeled with a second fluorophore that is of a different “color” compared to the first fluorophore) are flowed in the flow cell to interrogate the next base (e.g., base “A” at the 5′ of the base “T” in the template molecules). In a particular cycle, a mixture of dye-labeled nucleotides may be introduced into the flow cell, e.g., four fluorescent dyes each of a different “color” may be used to label A, T, C, and G, respectively (such as in a 4-channel SBS chemistry) or two different fluorescent dyes may be used (e.g., in a 2-channel SBS chemistry using “red” for C, “green” for T, “red” and “green” appearing as “yellow” for A, and unlabeled for G). Regardless, these known SBS methods require deactivation of fluorescent signals, e.g., via cleavage of fluorescently labeled reversible terminators on incorporated nucleotides, in order to allow incorporation of nucleotides to interrogate the next base. One or more washes between flow cell cycles are also performed, e.g., in order to remove unincorporated nucleotides and/or cleaved fluorescent labels. These and other requirements of known SBS methods have kept their costs high (e.g., as compared to methods based on qPCR or antigen-antibody interactions) and have limited their applications, especially in sequencing-based diagnostics, for instance, in response to a pandemic such as COVID-19 where large numbers of samples must be sequenced in a short period of time.

In some embodiments, provided herein is a method for nucleic acid sequencing, comprising: a) contacting a cluster immobilized on a substrate with a primer and terminated nucleotide molecules which may but do not need to be detectably labeled, wherein the cluster comprises nucleic acid molecules each comprising a common nucleic acid sequence to be sequenced, and wherein at a first subset of the nucleic acid molecules in the cluster, a terminated nucleotide molecule is incorporated into the primer hybridized to each nucleic acid molecule in the first subset using the nucleic acid molecule as template, thereby deactivating the nucleic acid molecule by preventing phosphodiester bond formation of a nucleotide with the incorporated terminated nucleotide molecule, whereas a second subset of the nucleic acid molecules in the cluster are not deactivated. In some embodiments, the method further comprises b) contacting the cluster with a plurality of nucleotides comprising detectably labeled nucleotide molecules, wherein nucleotides are not incorporated at the deactivated nucleic acid molecules in the first subset, and a detectably labeled nucleotide molecule is incorporated at a non-deactivated nucleic acid molecule in the second subset using the non-deactivated nucleic acid molecule as template. In some embodiments, the method further comprises detecting signals associated with the incorporation of detectably labeled nucleotide molecules at individual nucleic acid molecules in the cluster, thereby determining a sequence of the common nucleic acid sequence to be sequenced.

In any of the embodiments herein, the detectably labeled nucleotide molecule can be incorporated into the primer or an extension product thereof hybridized to the non-deactivated nucleic acid molecule in the second subset using the non-deactivated nucleic acid molecule as template.

In any of the embodiments herein, a plurality of clusters can be immobilized on the substrate, each comprising clonal copies of a common nucleic acid sequence to be sequenced.

In any of the embodiments herein, at least 100, at least 1,000, at least 10,000, at least 100,000, at least 1,000,000, or more than 1,000,000 clusters can be immobilized on the substrate. In any of the embodiments herein, at least 100, at least 1,000, at least 10,000, at least 100,000, at least 1,000,000, or more than 1,000,000 different common nucleic acid sequences to be sequenced can be in the clusters immobilized on the substrate.

In any of the embodiments herein, the terminated nucleotide molecules can comprise A nucleotides, T/U nucleotides, C nucleotides, and/or G nucleotides. In any of the embodiments herein, the terminated nucleotide molecules can contain only one, only two, only three, or all four of A nucleotides, T/U nucleotides, C nucleotides, and G nucleotides. In any of the embodiments herein, the cluster can be contacted with a plurality of nucleotide molecules comprising the terminated nucleotide molecules and non-terminated nucleotide molecules.

In any of the embodiments herein, the non-terminated nucleotide molecules can comprise A nucleotides, T/U nucleotides, C nucleotides, and/or G nucleotides. In any of the embodiments herein, the non-terminated nucleotide molecules can contain only one, only two, only three, or all four of A nucleotides, T/U nucleotides, C nucleotides, and G nucleotides. In any of the embodiments herein, the plurality of nucleotide molecules can comprise: i) terminated A nucleotide molecules and non-terminated A nucleotide molecules; ii) terminated T/U nucleotide molecules and non-terminated T/U nucleotide molecules; iii) terminated C nucleotide molecules and non-terminated C nucleotide molecules; and/or iv) terminated G nucleotide molecules and non-terminated G nucleotide molecules.

In any of the embodiments herein, the ratio of terminated nucleotide molecules to non-terminated nucleotide molecules in the plurality of nucleotide molecules can be at least or about 1:10, at least or about 1:8, at least or about 1:6, at least or about 1:4, at least or about 1:2, at least or about 1:1, at least or about 2:1, at least or about 4:1, at least or about 6:1, at least or about 8:1, at least or about 10:1, at least or about 20:1, at least or about 50:1, at least or about 100:1, at least or about 200:1, or at least or about 500:1.

In any of the embodiments herein, the ratio of terminated A nucleotide molecules to non-terminated A nucleotide molecules can be between about 1:4 and about 4:1; the ratio of terminated T/U nucleotide molecules to non-terminated T/U nucleotide molecules can be between about 1:4 and about 4:1; the ratio of terminated C nucleotide molecules to non-terminated C nucleotide molecules can be between about 1:4 and about 4:1; and/or the ratio of terminated G nucleotide molecules to non-terminated G nucleotide molecules can be between about 1:4 and about 4:1.

In any of the embodiments herein, the cluster can be contacted with: i) terminated A nucleotide molecules and non-terminated A nucleotide molecules, ii) terminated T/U nucleotide molecules and non-terminated T/U nucleotide molecules, iii) terminated C nucleotide molecules and non-terminated C nucleotide molecules, and iv) terminated G nucleotide molecules and non-terminated G nucleotide molecules. In some embodiments, the cluster is contacted with any two, any three, or all four of i), ii), iii), and iv) pre-mixed in a mixture. In some embodiments, the cluster is contacted with i), ii), iii), and iv) sequentially in separate cycles.

In any of the embodiments herein, the terminated nucleotide molecules can comprise irreversibly terminated nucleotide molecules. In any of the embodiments herein, the terminated nucleotide molecules can comprise ddNTP. In any of the embodiments herein, the terminated nucleotide molecules can comprise reversibly terminated nucleotide molecules. In some embodiments, the method does not comprise removing a reversible terminating group to render the reversibly terminated nucleotide molecules capable of forming phosphodiester bonds after incorporation of the reversibly terminated nucleotide molecules.

In any of the embodiments herein, the ratio of the number of molecules in the first subset to that in the second subset can be at least or about 1:10, at least or about 1:8, at least or about 1:6, at least or about 1:4, at least or about 1:2, at least or about 1:1, at least or about 2:1, at least or about 4:1, at least or about 6:1, at least or about 8:1, at least or about 10:1, at least or about 20:1, at least or about 50:1, at least or about 100:1, at least or about 200:1, or at least or about 500:1. In any of the embodiments herein, the ratio of the number of molecules in the first subset to that in the second subset can be between about 1:4 and about 4:1.

In any of the embodiments herein, the density of non-deactivated nucleic acid molecules in the cluster can be one molecule per at least about 250 nm², one molecule per at least about 200 nm², one molecule per at least about 150 nm², one molecule per at least about 100 nm², one molecule per at least about 50 nm², or one molecule per at least about 20 nm², or any value in between the aforementioned values.

In any of the embodiments herein, the detectably labeled nucleotide molecules can comprise the same detectable label. In any of the embodiments herein, the detectably labeled nucleotide molecules can comprise two, three, four, or more different detectable labels. In any of the embodiments herein, among the detectably labeled nucleotide molecules, two or more nucleotides comprising the same base can be labeled with different detectable labels, and/or two or more nucleotides comprising different bases can be labeled with the same detectable label.

In any of the embodiments herein, among the detectably labeled nucleotide molecules, nucleotides comprising the same base can be labeled with the same detectable label, and nucleotides comprising different bases can be labeled with different detectable labels each corresponding to a different base, optionally wherein A, T/U, C, and G each corresponds to a fluorophore identifying the base from among the four bases.

In any of the embodiments herein, the primer can hybridize to the nucleic acid molecule at a sequence that is 3′ to the common nucleic acid sequence to be sequenced. In any of the embodiments herein, the detectably labeled nucleotide molecules can be incorporated using the common nucleic acid sequence to be sequenced as template, thereby determining the sequence of the common nucleic acid sequence. In any of the embodiments herein, the non-deactivated nucleic acid molecules are sequenced using a single molecule real-time sequencing method.

In any of the embodiments herein, the substrate can comprise a bead, a planar substrate, a solid surface, a flow cell, a semiconductor chip, a well (optionally a microwell), a pillar (optionally a micropillar), a chamber (optionally a microchamber), a channel (optionally a microchannel), a through hole, a nanopore, or any combination thereof. In any of the embodiments herein, the nucleic acid molecules can comprise DNA and/or RNA.

In any of the embodiments herein, the plurality of nucleotides contacted with the cluster can comprise nucleotide molecules that are non-terminated and nucleotide molecules that are reversibly terminated. In any of the embodiments herein, the non-terminated nucleotide molecules can comprise detectably labeled nucleotide molecules and/or non-detectably labeled nucleotide molecules. In any of the embodiments herein, the reversibly terminated nucleotide molecules can comprise detectably labeled nucleotide molecules and/or non-detectably labeled nucleotide molecules. In any of the embodiments herein, the non-terminated nucleotide molecules can be non-detectably labeled, and the reversibly terminated nucleotide molecules can be detectably labeled. In any of the embodiments herein, the non-terminated nucleotide molecules can be detectably labeled, and the reversibly terminated nucleotide molecules can be non-detectably labeled. In any of the embodiments herein, the reversibly terminated nucleotide molecules can be incorporated and terminate stochastically at nucleic acid molecules in the cluster, thereby increasing phasing among non-deactivated nucleic acid molecules compared to that among non-deactivated nucleic acid molecules contacted with only non-terminated nucleotide molecules or with only reversibly terminated nucleotide molecules for sequencing.

In any of the embodiments herein, the cluster can be contacted with a plurality of primers each comprising a sequence complementary to a different region in the common nucleic acid sequence to be sequenced. In any of the embodiments herein, a terminated nucleotide molecule is incorporated into at least some of the plurality of primers hybridized in or adjacent to the common nucleic acid sequence to be sequenced. In any of the embodiments herein, the method can comprise determining a sequence of the common nucleic acid sequence using at least some of the different primers hybridized to the non-deactivated nucleic acid molecules in the cluster. In any of the embodiments herein, the sequences determined using the different primers can be analyzed using multiple alignment. In any of the embodiments herein, the sequences determined using the different primers can be synthesized to form a synthetic long read sequence of at least or about 100, at least or about 200, at least or about 500, at least or about 1,000, at least or about 2,000, or at least or about 5,000 nucleotides in length.

In any of the embodiments herein, the signals associated with the incorporation of detectably labeled nucleotide molecules can be detected using a total internal reflection fluorescence (TIRF) imaging system. In any of the embodiments herein, the TIRF imaging system can comprise a prism comprising a low auto-florescence plastic material. In any of the embodiments herein, the prism can be used as at least a portion of the substrate. In any of the embodiments herein, the TIRF imaging system can comprise an excitation filter below and/or above the substrate.

In some embodiments, provided herein are nucleic acid sequencing methods in which nucleotides that are sequentially incorporated (e.g., into a sequencing primer in the 5′ to 3′ direction) do not need to be cyclically introduced (e.g., into a flow cell that contains a sequencing reaction mix) and/or cyclically contacted with the template nucleic acid to be sequenced and a sequencing primer hybridized thereto, although in certain aspects such cyclic sequencing reactions may be performed. In some embodiments, real-time signals and/or changes thereof are detected as nucleotides are incorporated and/or their associated signals are deactivated, and since no cycles are required, there is no need to remove unincorporated nucleotides and/or cleaved labels (e.g., by one or more washes), although in certain aspects such removing steps may be performed.

In some embodiments, a first labeled nucleotide that has been incorporated is not deactivated (e.g., by removal and/or photobleaching of the label) prior to the introduction and/or incorporation of the next, second labeled nucleotide. The first and second labeled nucleotides can comprise the same base or different bases. The first and second labeled nucleotides can be introduced into a sequencing reaction mix simultaneously or at different time points in any order. Further, the first and second labeled nucleotides can be introduced by itself (e.g., in a suitable solvent such as water) or in a mixture with another sequencing reagent, such as one or more other labeled nucleotides and/or one or more unlabeled nucleotides. The first and second labeled nucleotides can also comprise the same base or different bases. In some embodiments, nucleotides that have not been incorporated at a residue corresponding to a base in the template nucleic acid (e.g., because the first labeled nucleotide has been incorporated at that residue) are not removed from the sequencing reaction mix prior to the introduction and/or incorporation of the second labeled nucleotide. In some embodiments, the first and second labeled nucleotides (and optionally labeled nucleotides for interrogating subsequent bases in the template) are provided in the same sequencing reaction mix, and the first, second, and optionally any subsequent labeled nucleotide(s) are incorporated sequentially in a continuous manner. Thus, unlike existing SBS methods, some embodiments of the method disclosed herein use continuous introduction and/or incorporation of nucleotides (e.g., fluorescently labeled A, T, C, and/or G nucleotides) without the need of label deactivation and/or wash steps in between sequential incorporation events for a given template nucleic acid molecule to be sequenced. Rather, in some embodiments, label deactivation (e.g., by cleaving and/or photobleaching the label) of a first incorporated nucleotide may occur stochastically throughout the continuous nucleotide incorporation process, for instance, prior to, during, or after the incorporation of a second, third, fourth, or a subsequent labeled nucleotide.

In contrast to the example of known SBS methods above, in one embodiment of a method disclosed herein, dye-labeled “A” nucleotides incorporated at the particular spots are not completely deactivated (e.g., by cleavage and removal of fluorescently labeled reversible terminators) prior to the addition of dye-labeled “T” nucleotides. Instead, the incorporated nucleotides may be stochastically deactivated (e.g., by photobleaching and/or cleaving the labels) in a non-cyclically manner. In other words, signals associated with incorporated nucleotides at multiple different spots in a flow cell do not need to be deactivated in the same cycle or in a synchronized manner. In some embodiments, incorporated nucleotides at two or more different spots are illuminated using the same light (e.g., excitation light of the same wavelength). In some embodiments, incorporated nucleotides at two or more different spots are each illuminated using a different light (e.g., excitation light of a different wavelength). Referring to the example, a laser can be used to illuminate the dyes on the incorporated “A” nucleotides, which with some probability will be bleached, e.g., by the same laser that is used to illuminate. It is not essential whether the dyes on the incorporated “A” nucleotides are in fact deactivated by photobleaching (or not deactivated), as the signals (e.g., signal intensity) may be deconvoluted to detect such stochastic deactivation events. Dye-labeled “T” nucleotides can be provided together with (e.g., in the same mixture) dye-labeled “A” nucleotides that have incorporated and/or those yet to be incorporated, and signals associated with the “A” and “T” nucleotides at the particular spots can be monitored over time. In some embodiments, the dye-labeled “T” nucleotides can (but do not need to) be introduced after the dye-labeled “A” nucleotides are introduced (e.g., into a flow cell) and some of the “A” nucleotides are incorporated into primer strands at various locations. During the sequencing process, e.g., during or after incorporation of a dye-labeled “T” nucleotide at a particular spot, the previously incorporated dye-labeled “A” nucleotide can bleach out. Thus, there is no requirement of bleaching of all dye-labeled “A” nucleotides before introducing more dye-labeled nucleotides (dye-labeled “T” nucleotides in this example), and in fact, dye-labeled “A” nucleotides that have not incorporated can remain in the sequencing reaction mix such that they can be incorporated when one or more complementary bases in the template after the “A” (which base pairs with the dye-labeled “T” nucleotide incorporated in the sequencing primer) is again “T.” For instance, a mixture of dye-labeled “A” nucleotides and dye-labeled “T” nucleotides can be used to sequence “TAT” in a template without complete signal deactivation of an incorporated nucleotide and/or removal of any unincorporated nucleotide.

In some embodiments, stepwise changes over time in fluorophore emission (e.g., stepwise increases and/or decreases in signal intensity) at the particular spots can be detected and/or monitored. An increase in signal intensity (e.g., due to a nucleotide incorporation) and/or a decrease in signal (e.g., due to a photobleaching event) at a particular spot and in a given time window or time point (e.g., an imaging window in terms of frame/exposure) may partially or completely offset one another. In some embodiments, incorporation of a labeled nucleotide results in an increase in signal intensity characteristic of the label and/or the base of the incorporated labeled nucleotide. For instance, a nucleotide can be labeled with a label having a signal intensity characteristic of the base in that nucleotide, which can be distinguished from the signal intensity of the label on another nucleotide having a different base. In some embodiments, signal deactivation (e.g., by cleaving and/or photobleaching the label) of a labeled nucleotide results in a decrease in signal intensity characteristic of the label and/or the base of the signal-deactivated labeled nucleotide.

In some embodiments, for a given labeled nucleotide, once the label is cleaved or deactivated, the signal intensity (if any remains) associated with the nucleotide no longer changes, e.g., in response to light that bleaches labels on other nucleotides. For instance, in one embodiment, after the fluorescent dye of a particular dye-labeled nucleotide is photobleached (thus fluorescence intensity associated with dye-labeled nucleotide decreases from a first intensity to a second, lower intensity), the photobleached dye-labeled nucleotide does not recover to the first fluorescence intensity. In some embodiments, the fluorescence intensity of the photobleached dye-labeled nucleotide remains at the second intensity which can be zero; in other words, the photobleached dye can go “dark,” e.g., its signal is below a certain threshold or undetectable and does not recover. In some embodiments, an increase in signal intensity due to a nucleotide incorporation event in a method disclosed herein is not detected as an increase due to a photobleached dye recovering from a bleached state. In some embodiments, a photobleached dye herein is prevented from recovering from a bleached state such that an increase in signal intensity is attributable to nucleotide incorporation rather than recovery from photobleaching. In some embodiments, for each label that has been deactivated (e.g., photobleached), the deactivation is complete in that the deactivated label does not recover. In some embodiments, labels at multiple locations (some of which may comprise the same label and others may comprise different labels) are not deactivated (e.g., photobleached) at the same time or in the same time window (e.g., in the same cycle). Rather, in a method disclosed herein, labels at different locations may be deactivated stochastically such that at a given time point or in a given time window, the labels at all locations of the substrate are not completely deactivated whereas for each label the signal deactivation is or will be complete (e.g., no signal recovery from a deactivated state).

In some embodiments where recovery from a deactivated state (e.g., after photobleaching) may occur, a recovery probability may be modeled and used during base calling. In some embodiments, the recovery probability is modeled using a reference based correction. Dye recovery from photobleaching has been described, for instance, by Braslavsky et al., “Sequence information can be obtained from single DNA molecules,” PNAS 100(7): 3960-64 (2003), incorporated herein by reference in its entirety for all purposes.

In some embodiments, the net change in signal intensity at the particular spot and the given time window or time point can be associated with the event(s) at the particular spot, for instance, incorporation of a new labeled nucleotide and photobleaching of one or more already incorporated labeled nucleotides. The one or more already incorporated labeled nucleotides may be at any distance from the newly incorporated labeled nucleotide, e.g., 0, 1, 2, 3, 4, 5, or more nucleotide residues apart. In some embodiments, the net change in signal intensity may be deconvoluted to one or more increases and/or one or more decreases in signal intensity that are characteristic of a nucleotide incorporation event (e.g., incorporation of a nucleotide labeled with a particular fluorophore) and a signal deactivation event (e.g., photobleaching of the same or another particular fluorophore), respectively.

In some embodiments, provided herein is a method for determining a sequence of a nucleic acid molecule, comprising contacting the nucleic acid molecule with an enzyme capable of templated nucleic acid polymerization, such as a polymerase (e.g., a DNA-dependent DNA polymerase or an RNA-dependent DNA polymerase), a first detectably labeled nucleotide, and a second detectably labeled nucleotide. In any of the embodiments herein, the contacting step can be non-cyclic. In any of the embodiments herein, the first and second detectably labeled nucleotides can be complementary to adjacent nucleotides in the nucleic acid molecule, and would have to be incorporated in separate flow cell cycles in some existing cyclic sequencing methods (that is, the first detectably labeled nucleotide would have to be incorporated in a first cycle, unincorporated detectably labeled nucleotides in the first cycle would have to be removed, signals of the detectably labeled nucleotides incorporated in the first cycle would have to be deactivated and/or removed, and only after that the second detectably labeled nucleotide would be contacted with the nucleic acid molecule and/or a sequencing primer hybridized thereto in a second cycle of nucleotide incorporation, washing, and signal deactivation and/or removal).

In any of the embodiments herein, the nucleic acid molecule (the template) can be contacted with the first and second detectably labeled nucleotides in the same reaction mix. Alternatively, in any of the embodiments herein, the nucleic acid molecule can be contacted with the first detectably labeled nucleotide and then the second detectably labeled nucleotide, or with the second detectably labeled nucleotide and then the first detectably labeled nucleotide.

In any of the embodiments herein, the nucleic acid molecule can be hybridized to a primer. In any of the embodiments herein, the polymerase, the nucleic acid molecule, and/or the primer can be immobilized at a location (a “spot”) of a substrate (e.g., a chamber having a planar surface, such as one that can be used for a single molecule, real-time sequencing reaction and detection). In some aspects, the nucleic acid molecule is directly or indirectly attached to the substrate, and the attachment can comprise covalent attachment (e.g., by one or more covalent bonds) and/or noncovalently attachment (e.g., via one or more binding pairs such as biotin/streptavidin binding). The immobilized nucleic acid molecule may capture the primer which can be provided in a sequencing reaction mix, e.g., together with the polymerase and/or the first and/or second detectably labeled nucleotides. In some aspects, the primer is directly or indirectly attached to the substrate, and the attachment can comprise covalent attachment (e.g., by one or more covalent bonds) and/or noncovalently attachment (e.g., via one or more binding pairs such as biotin/streptavidin binding). The immobilized primer may capture the nucleic acid molecule to be sequenced, which can be provided in a sequencing reaction mix, e.g., together with the polymerase and/or the first and/or second detectably labeled nucleotides. In some aspects, the polymerase is directly or indirectly attached to the substrate, and the attachment can comprise covalent attachment (e.g., by one or more covalent bonds) and/or noncovalently attachment (e.g., via one or more binding pairs such as biotin/streptavidin binding and/or antibody/antigen binding). The immobilized polymerase may capture the nucleic acid molecule to be sequenced and/or the primer, which can be provided in a sequencing reaction mix, e.g., together with the first and/or second detectably labeled nucleotides. In some embodiments, any two or more of the polymerase, the nucleic acid molecule, and the primer can be immobilized. Alternatively, in some embodiments, only one of the polymerase, the nucleic acid molecule, and the primer is immobilized. In some embodiments, at a first location a polymerase is immobilized to the substrate while a nucleic acid molecule to be sequenced and/or a sequencing primer are provided in a reaction mix (e.g., solution); at a second location a nucleic acid molecule to be sequenced is immobilized to the substrate while a polymerase and/or a sequencing primer are provided in a reaction mix (e.g., solution); and/or at a third location a sequencing primer is immobilized to the substrate while a polymerase and/or a nucleic acid molecule to be sequenced are provided in a reaction mix (e.g., solution). Sequencing reactions at the first, second, third locations can proceed in parallel or in any suitable order, and utilize the same polymerase or different polymerases.

In any of the embodiments herein, polymerase molecules, nucleic acid molecules to be sequenced, and/or sequencing primer molecules can be randomly attached to locations on the substrate. Alternatively, in any of the embodiments herein, polymerase molecules, nucleic acid molecules to be sequenced, and/or sequencing primer molecules can be attached to locations on the substrate in an ordered way, for instance, the molecules can be arrayed according to a pattern which may be predetermined. In any of the embodiments herein, polymerase molecules, nucleic acid molecules to be sequenced, and/or sequencing primer molecules can be attached to locations on the substrate in a controlled manner, e.g., at a particular density of molecules per unit area of the substrate. In any of the embodiments herein, the distances between adjacent polymerase molecules, nucleic acid molecules to be sequenced, and/or sequencing primer molecules on the substrate are such that signals (e.g., optical signals such as fluorescence) associated with and/or indicative of reactions at adjacent molecules can be spatially and/or optically resolved, e.g., at a single molecule resolution.

In any of the embodiments herein, the sequencing reaction at an individual location (e.g., spot) on the substrate can occur and be analyzed at a single molecule level. In any of the embodiments herein, signals at an individual location (e.g., a spot having a single template nucleic acid molecule immobilized thereto) on the substrate can be monitored over time. In any of the embodiments herein, signals detected over time a particular location can be associated with and/or indicative of events occurring on a single nucleic acid molecule to be sequenced and/or a single sequencing primer at the particular location. In any of the embodiments herein, an individual location (e.g., spot) on the substrate can comprise two or more copies of a nucleic acid molecule to be sequenced, for instance, clonal copies of the nucleic acid molecule. In cases where a spot comprises copies of the nucleic acid molecule, any suitable cyclic SBS reactions including those known in the art may be used in combination with a method disclosed herein. Alternatively, in any of the embodiments herein, a single molecule of a nucleic acid to be sequenced and/or a sequencing primer hybridized there to can be attached to each individual location (e.g., spot) on the substrate.

In any of the embodiments herein, the first detectably labeled nucleotide can be complementary to a first nucleotide of the nucleic acid molecule and thus can be incorporated into the primer by the polymerase, thereby generating an extended primer comprising the incorporated first detectably labeled nucleotide at the location. In some embodiments, the incorporated first detectably labeled nucleotide is the 3′ terminal nucleotide of the extended primer, although the first nucleotide can be an internal nucleotide in the nucleic acid molecule to be sequenced.

In any of the embodiments herein, the second detectably labeled nucleotide can be complementary to a second nucleotide of the nucleic acid molecule and thus can be incorporated into the extended primer by the polymerase, thereby generating a further extended primer comprising the incorporated first and second detectably labeled nucleotides at the location. In some embodiments, the incorporated second detectably labeled nucleotide forms a phosphodiester bond with the incorporated first detectably labeled nucleotide and is the 3′ terminal nucleotide of the further extended primer. The second nucleotide can be an internal nucleotide and can be 5′ to the first nucleotide in the nucleic acid molecule to be sequenced.

In any of the embodiments herein, multiple labels may be emitting at a given time point or in a time window. For instance, the detectable label of an incorporated nucleotide and the detectable label of another incorporated nucleotide can be emitting at the same time point or in the same time window, and the first and second incorporated nucleotides can be immediately adjacent (e.g., connected directly by a phosphodiester bond) or one, two, three, or more nucleotide residues from each other in the strand comprising the sequencing primer. In some embodiments, one or more detectably labeled nucleotides have been incorporated in the sequencing primer at a given substrate location and are emitting when a subsequent detectably labeled nucleotide is incorporated. In some embodiments, signals of the detectably labeled nucleotides incorporated at the substrate location can be detected in the same detection channel, such as a fluorescent channel of a fluorescence microscope, and signals of different detectable labels are detected (“observed”) simultaneously (e.g., in the same detection channel by the same camera), where signal intensities of different detectable labels can be combined. For instance, at the same detection time point or in the same detection window, an increase in signal intensity due to incorporation of a first labeled nucleotide (e.g., “A” labeled with ATTO 532) and an increase in signal intensity due to incorporation of a second labeled nucleotide (e.g., “T” labeled with ATTO 542) in the same primer strand can be added to provide a combined signal intensity value for the detection time point or window. Likewise, at the same detection time point or in the same detection window, a decrease in signal intensity due to photobleaching of the first label (e.g., ATTO 532 on an incorporated “A” nucleotide) and a decrease in signal intensity due to photobleaching of the a second label (e.g., by ATTO 542 on an incorporated “T” nucleotide) in the same primer strand can be added, whereas an increase due to incorporation and a decrease due to photobleaching may at least partially offset one another. In some embodiments, a method disclosed herein does not use more than one light filters, e.g., switchable filters. In some embodiments, a method disclosed herein detects signals of different detectable labels simultaneously, and a filter (e.g., a dichroic filter) can be used to split emissions from the substrate location into separate channels each detectable by a separate camera. Each camera may be used detect light in a different detection channel (“color”) and a plurality of different detection channels can be used in a method disclosed herein. As such, signals associated with different detectable labels can be detected (“observed”) simultaneously. In some embodiments, signal intensities (e.g., the sum of relative fluorescence over a range of wavelengths) of different detectable labels detected at different detection channels and/or at different cameras can be combined, and optionally a change in signal intensity of one detectable label (e.g., ATTO 532) can be compared to and/or combined with a change in signal intensity of a different detectable label (e.g., ATTO 542).

In any of the embodiments herein, a method disclosed herein can further comprise deactivating the detectable label(s) of the incorporated first and/or second detectably labeled nucleotides. In any of the embodiments herein, the detectable label of the incorporated first detectably labeled nucleotide can be deactivated prior to, during, and/or after the incorporation of the second detectably labeled nucleotide. In some embodiments, the detectable label of the incorporated first detectably labeled nucleotide is deactivated prior to the incorporation of the second detectably labeled nucleotide. In some embodiments, the detectable label of the incorporated first detectably labeled nucleotide is deactivated during the incorporation of the second detectably labeled nucleotide. In some embodiments, the detectable label of the incorporated first detectably labeled nucleotide is deactivated after the incorporation of the second detectably labeled nucleotide, for instance, immediately after the incorporation of the second detectably labeled nucleotide or after the incorporation of a third, fourth, or subsequent detectably labeled nucleotide.

In any of the embodiments herein, deactivation of a particular detectable label at a particular substrate location can occur stochastically. In some embodiments, the deactivation of a particular detectable label and/or the timing of such deactivation is not preselected or predetermined. In some embodiments, the deactivation of a particular detectable label and/or the timing of such deactivation is not cyclic. In any of the embodiments herein, deactivation of the detectable labels at different substrate locations are not synchronized. In any of the embodiments herein, deactivation of the detectable labels at different substrate locations and/or the timing of such deactivation events are stochastic and not according to a preselected or predetermined scheme or pattern. In some embodiments, deactivation of the detectable labels at different substrate locations is not performed in one cycle or in sequential cycles.

In any of the embodiments herein, the method can further comprise detecting signals or absence thereof associated with the detectable labels at the location over time, thereby generating a time trace of signal intensity associated with the incorporation and/or detectable label deactivation of detectably labeled nucleotides at the location. In any of the embodiments herein, the method can further comprise using the time trace to identify the first and second detectably labeled nucleotides, thereby determining a sequence comprising the first and second nucleotides of the nucleic acid molecule.

In any of the embodiments herein, the contacting step can comprise contacting the nucleic acid molecule with the polymerase, the first detectably labeled nucleotide, the second detectably labeled nucleotide, and a third detectably labeled nucleotide, wherein the third detectably labeled nucleotide is complementary to a third nucleotide in the nucleic acid molecule and is incorporated into the further extended primer by the polymerase, thereby generating a still further extended primer comprising the incorporated first, second, and third detectably labeled nucleotides at the location. The third nucleotide can be 5′ to the second nucleotide which in turn can be 5′ to the first nucleotide in the nucleic acid molecule.

In any of the embodiments herein, the deactivating step can comprise deactivating the detectable label(s) of the incorporated first, second, and/or third detectably labeled nucleotides. The detectable label of the incorporated second detectably labeled nucleotide can be deactivated prior to, during, and/or after the incorporation of the third detectably labeled nucleotide. Similarly, the detectable label of the incorporated first detectably labeled nucleotide can be deactivated prior to, during, and/or after the incorporation of the third detectably labeled nucleotide. Further, the detectable label of the incorporated first detectably labeled nucleotide can be deactivated prior to, during, and/or after the incorporation of the second detectably labeled nucleotide.

In any of the embodiments herein, the method can comprise using the time trace to identify the first, second, and third detectably labeled nucleotides, thereby determining a sequence comprising the first, second, and third nucleotides of the nucleic acid molecule.

In any of the embodiments herein, the contacting step can comprise contacting the nucleic acid molecule with the polymerase, the first detectably labeled nucleotide, the second detectably labeled nucleotide, the third detectably labeled nucleotide, and a fourth detectably labeled nucleotide, wherein the fourth detectably labeled nucleotide is complementary to a fourth nucleotide in the nucleic acid molecule and is incorporated into the still further extended primer by the polymerase, thereby generating a yet still further extended primer comprising the incorporated first, second, third, and fourth detectably labeled nucleotides at the location. The fourth nucleotide can be 5′ to the third nucleotide third nucleotide, which can be 5′ to the second nucleotide which in turn can be 5′ to the first nucleotide in the nucleic acid molecule.

In any of the embodiments herein, the deactivating step can comprise deactivating the detectable label(s) of the incorporated first, second, third, and/or fourth detectably labeled nucleotides. The detectable label of the incorporated third detectably labeled nucleotide can be deactivated prior to, during, and/or after the incorporation of the fourth detectably labeled nucleotide. The detectable label of the incorporated second detectably labeled nucleotide can be deactivated prior to, during, and/or after the incorporation of the fourth detectably labeled nucleotide. Similarly, the detectable label of the incorporated first detectably labeled nucleotide can be deactivated prior to, during, and/or after the incorporation of the fourth detectably labeled nucleotide. Further, the detectable label of the incorporated first detectably labeled nucleotide can be deactivated prior to, during, and/or after the incorporation of the second detectably labeled nucleotide. The detectable label of the incorporated second detectably labeled nucleotide can be deactivated prior to, during, and/or after the incorporation of the third detectably labeled nucleotide.

In any of the embodiments herein, the method can comprise using the time trace to identify the first, second, third, and fourth detectably labeled nucleotides, thereby determining a sequence comprising the first, second, third, and fourth nucleotides of the nucleic acid molecule.

In any of the embodiments herein, the nucleic acid molecule can comprise a deoxyribonucleotide or derivative or analog thereof and/or a ribonucleotide or derivative or analog thereof. In any of the embodiments herein, the nucleic acid molecule can comprise DNA or RNA. In any of the embodiments herein, a method disclosed herein can be used for direct RNA sequencing without first converting RNA to DNA such as cDNA.

In any of the embodiments herein, the polymerase can be a DNA-dependent polymerase and/or an RNA-dependent polymerase. In any of the embodiments herein, the same polymerase can be used to catalyze multiple nucleotide incorporation events using the same nucleic acid molecule as template. In any of the embodiments herein, the same polymerase can be used to catalyze multiple nucleotide incorporation events using different nucleic acid molecules as template, and the different nucleic acid molecules may be provided on substrate for single molecule sequencing. In any of the embodiments herein, different polymerases can be used to catalyze two or more nucleotide incorporation events using the same nucleic acid molecule as template. In any of the embodiments herein, different polymerases can be used to catalyze two or more nucleotide incorporation events using different nucleic acid molecules as template, and the different nucleic acid molecules may be provided on substrate for single molecule sequencing. In any of the embodiments herein, the rate(s) of nucleotide incorporation by the one or more polymerases can be controlled.

In any of the embodiments herein, the one or more polymerases can comprise a DNA polymerase and/or an RNA polymerase. In any of the embodiments herein, the polymerase can have a DNA-dependent DNA polymerase activity and/or an RNA-dependent DNA polymerase activity. In any of the embodiments herein, the one or more polymerases can be selected from the group consisting of DNA polymerase I, Klenow fragment of DNA polymerase I, DNA polymerase III, Taq polymerase, KlenTaq polymerase, TopoTaq polymerase, Bst polymerase, rBST DNA polymerase, Bsu polymerase, T7 DNA polymerase, T7 RNA polymerase, T3 DNA polymerase, T3 RNA polymerase, T4 polymerase, T5 polymerase, φ29 polymerase, 9° N polymerase, KOD polymerase, Pfu DNA polymerase, Vent DNA polymerase, Deep Vent DNA polymerase, Vent (exo-) polymerase, M2 polymerase, B103 polymerase, GA-1 polymerase, pPRD1 polymerase, N29 DNA polymerase, SP6 RNA polymerase, a reverse transcriptase (optionally a SuperScript® III reverse transcriptase), and a variant or derivative thereof.

In any of the embodiments herein, the one or more polymerases may not be immobilized to the substrate. In any of the embodiments herein, the one or more polymerases may not be immobilized at or near the substrate location which contains the nucleic acid molecule and/or the primer (e.g., sequencing primer). In any of the embodiments herein, the nucleic acid molecule can be immobilized at the location of the substrate. In any of the embodiments herein, the primer can be immobilized at the location of the substrate.

In any of the embodiments herein, the primer can comprise a deoxyribonucleotide or derivative or analog thereof and/or a ribonucleotide or derivative or analog thereof. In any of the embodiments herein, the primer can be protected from 3′-5′ exonuclease degradation by the polymerase while allowing 5′-3′ extension by the polymerase.

In any of the embodiments herein, the primer may not have been extended by one or more polymerases prior to step a). Alternatively, in any of the embodiments herein, the primer may have been extended by one or more polymerases prior to step a). In some embodiments, the primer may have been extended in nucleic acid sequencing comprising introducing nucleotides in one or more cycles and wash and/or signal deactivation following at least one cycle or between at least two consecutive cycles. For instance, an extended sequencing primer from cyclical sequencing-by-synthesis can be used in a method disclosed herein and be further extended to sequence additional bases in a nucleic acid molecule that the sequencing primer hybridizes to.

In any of the embodiments herein, the polymerase can have a 3′-5′ exonuclease activity, e.g., for proofreading. Alternatively, in any of the embodiments herein, the polymerase may lack a 3′-5′ exonuclease activity.

In any of the embodiments herein, the first, second, third, and/or fourth detectably labeled nucleotides can be independently selected from the group consisting of an ATP, a TTP, a CTP, a GTP, a UTP, a dATP, a dTTP, a dCTP, a dGTP, and a dUTP, and derivatives and analogs thereof. In any of the embodiments herein, the first, second, third, and/or fourth detectably labeled nucleotides can independently comprise a dATP, a dTTP, a dCTP, a dGTP, or a dUTP, or a derivative or analog thereof.

In any of the embodiments herein, each of the first, second, third, and fourth detectably labeled nucleotides can comprise a different base. In any of the embodiments herein, each of the first, second, third, and fourth detectably labeled nucleotides can independently comprise A, T, C, G, or U, or a derivative or analog thereof.

In any of the embodiments herein, any two, three, or all of the first, second, third, and fourth detectably labeled nucleotides can comprise the same base. The same base can be A, T, C, G, or U, or a derivative or analog thereof.

In any of the embodiments herein, any one, two, three, or all of the first, second, third, and fourth detectably labeled nucleotides can be void of a terminating group. In any of the embodiments herein, prior to nucleotide incorporation, any one, two, three, or all of the first, second, third, and fourth detectably labeled nucleotides can lack a reversible terminating group or an irreversible terminating group. In any of the embodiments herein, prior to nucleotide incorporation, any one, two, three, or all of the first, second, third, and fourth detectably labeled nucleotides can lack a 3′-O-blocking group and/or a detectable label that functions as a terminating group. In any of the embodiments herein, prior to nucleotide incorporation, any one, two, three, or all of the first, second, third, and fourth detectably labeled nucleotides can lack a dideoxynucleotide group.

In any of the embodiments herein, any one, two, three, or all of the first, second, third, and fourth detectably labeled nucleotides may comprise one or more molecules of the same detectable label or multiple different detectable labels. In some embodiments, each of the first, second, third, and fourth detectably labeled nucleotides comprises one detectable label. In other embodiments, any one, two, three, or all of the first, second, third, and fourth detectably labeled nucleotides can comprise two or more different detectable labels.

In any of the embodiments herein, among the detectably labeled nucleotides, two or more nucleotides comprising the same base can be labeled with different detectable labels. In any of the embodiments herein, two or more nucleotides comprising different bases may be labeled with the same detectable label.

In any of the embodiments herein, among the detectably labeled nucleotides, nucleotides comprising the same base can be labeled with the same detectable label, and nucleotides comprising different bases can be labeled with different detectable labels each corresponding to a different base. For instance, A, T, C, and G each can correspond to a fluorophore identifying the base from among the four bases.

In any of the embodiments herein, a method disclosed herein may comprise contacting the nucleic acid molecule with one or more unlabeled nucleotides which may or may not be incorporated.

In any of the embodiments herein, the detectable labels may comprise fluorophores having different emission wavelengths and/or fluorophores having different fluorescence intensity at the same emission wavelength and/or in the same region of emission wavelengths. For instance, a first base and a second base may correspond to a first fluorophore and a second fluorophore, respectively. In some aspects, the first and second bases are different and may be independently selected from the group consisting of A, T/U, C, and G. In some aspects, the fluorescence intensity of the first fluorophore is at least about 1.2, at least about 1.5, at least about 2, at least about 2.5, at least about 3, at least about 3.5, at least about 4, at least about 4.5, or at least about 5 times the fluorescence intensity of the second fluorophore at the same emission wavelength and/or in the same region of emission wavelengths, or vice versa. In some aspects, the total fluorescence intensity of one or more molecules of the first fluorophore is distinguishable from the total fluorescence intensity of one or more molecules of the second fluorophore.

In any of the embodiments herein, the increase in fluorescence intensity associated with the incorporation of one nucleotide molecule comprising the first base and the first fluorophore is distinguishable from the increase in fluorescence intensity associated with the incorporation of multiple nucleotide molecules each comprising the second base and the second fluorophore. In any of the embodiments herein, the increase in fluorescence intensity associated with the incorporation of one nucleotide molecule comprising the second base and the second fluorophore is distinguishable from the increase in fluorescence intensity associated with the incorporation of multiple nucleotide molecules each comprising the first base and the first fluorophore.

In any of the embodiments herein, the method can further comprise contacting the nucleic acid molecule with the primer. The primer can be hybridized prior to, during, or after immobilization of the nucleic acid molecule to the substrate. In any of the embodiments herein, the nucleic acid molecule can be contacted with the primer, the polymerase, and the first, second, third, and/or fourth detectably labeled nucleotides in any order, e.g., in order to provide a sequencing reaction mix comprising any two or more of the aforementioned reagents. In some embodiments, the sequencing reaction mix can comprise the nucleic acid molecule, the primer, and the polymerase, and the first, second, third, and/or fourth detectably labeled nucleotides can be added to initiate nucleotide incorporation. In some embodiments, the sequencing reaction mix can comprise the nucleic acid molecule, the primer, and the first, second, third, and/or fourth detectably labeled nucleotides, and the polymerase can be added to initiate nucleotide incorporation. In some embodiments, the sequencing reaction mix can comprise the nucleic acid molecule, the primer, and the first, second, third, and/or fourth detectably labeled nucleotides, and the polymerase can be added to initiate nucleotide incorporation. In any of the embodiments herein, the nucleic acid molecule can be contacted with any two or more of the primer, the polymerase, and the first, second, third, and/or fourth detectably labeled nucleotides simultaneously. In any of the embodiments herein, the nucleic acid molecule hybridized to the primer can be contacted with the polymerase and the first, second, third, and fourth detectably labeled nucleotides simultaneously. In any of the embodiments herein, the nucleic acid molecule hybridized to the primer can be first contacted with the polymerase, followed by contacting the first, second, third, and fourth detectably labeled nucleotides simultaneously.

In any of the embodiments herein, the first, second, third, and/or fourth nucleotides can be immediately adjacent to one another in the 3′ to 5′ direction in the nucleic acid molecule. In any of the embodiments herein, the nucleic acid molecule may be 3′ or 5′ immobilized on the substrate, via covalent linking or non-covalent linking (e.g., the primer can be immobilized to the substrate whereas the nucleic acid molecule hybridizes to the primer).

In any of the embodiments herein, a particular incorporation event can occur in the presence of detectably labeled nucleotide molecules that have not incorporated in one or more preceding incorporation events at an individual location. In any of the embodiments herein, the first, second, third, and fourth detectably labeled nucleotides can be continuously incorporated in the same reaction volume, e.g., without removing other detectably labeled nucleotides prior to, during, or after incorporation of any particular detectably labeled nucleotide from the reaction volume. In some embodiments, no detectably labeled nucleotide molecule is removed from the reaction volume by washing between two incorporation events using the same nucleic acid molecule being sequenced as template.

In any of the embodiments herein, one or more additional detectably labeled nucleotide molecules can be provided in the reaction volume. In some embodiments, a mixture of detectably labeled nucleotide molecules collectively comprising two, three, or four or more bases (e.g., A, T/U, C, and G) can be continuously introduced into the reaction volume. The mixture may comprise one or more other reagents including polymerase molecules and cofactors such as Mg²⁺.

In any of the embodiments herein, the method can further comprise controlling the rate of nucleotide incorporation during SBS, e.g., by controlling the temperature of the sequencing reaction. In any of the embodiments herein, the method can further comprise controlling the temperature of the reaction volume disclosed herein, such that the rate of nucleotide incorporation may be controlled.

In any of the embodiments herein, the method can further comprise the presence/absence or the amount(s) and/or concentration(s) of one or more incorporating nucleotides and/or one or more non-incorporating nucleotides in the reaction volume. In some embodiments, the one or more incorporating nucleotides comprise the first, second, third, and/or fourth detectably labeled nucleotides. In some embodiments, the one or more incorporating nucleotides comprise an NDP (e.g., ADP, TDP, UDP, CDP, or GDP), a dNDP (e.g., dADP, dTDP, dUDP, dCDP, or dGDP), or a derivative or analog thereof, or any combination thereof. In some embodiments, the one or more non-incorporating nucleotides comprise an NMP (e.g., AMP, TMP, UMP, CMP, or GMP), a dNMP (e.g., dAMP, dTMP, dUMP, dCMP, or dGMP), or a derivative or analog thereof, or any combination thereof. In some aspects, a non-incorporating nucleotide or analog thereof can transiently bind to a polymerase but is not incorporated by the polymerase. In some aspects, an incorporating nucleotide or analog thereof can be incorporated by a polymerase at a slower rate than a corresponding naturally-occurring nucleoside triphosphate (e.g., NTP or dNTP). As such, by adjusting the relative concentration(s) of non-incorporating nucleotide(s) and/or slower incorporating nucleotide(s) in the reaction volume, incorporation rates of one or more nucleotides during SBS may be fine-tuned as needed.

Certain divalent or trivalent metal cofactors such as magnesium and manganese are known to interact with a polymerase to modulate the progress of the reaction. Such catalytic metal cofactors can coordinate with a polymerase and the triphosphate of a dNTP to catalyze the addition of a nucleotide to the 3′ terminal nucleotide on the end of the initiator (e.g., a primer), creating a phosphodiester linkage between the nucleotide of the dNTP and the initiator and releases pyrophosphate (PPi). Other metal ions, such as Ca²⁺, can interact with a polymerase and stabilize the enzyme, thereby slowing down nucleotide incorporation. Different metal co-factors can have varying catalytic effects upon the polymerization reaction depending upon the nature of the polymerization reaction, the polymerase used, the nucleotides employed, etc. In some embodiments, absent the metal cofactor in the proper oxidation state, polymerization will not occur at an appreciable rate even if all other necessary components are present. Metal cofactor cations may include Co²⁺, Mn²⁺, Zn²⁺ and/or Mg²⁺. Exemplary cofactor cations are disclosed in Vashishtha et al., J Biol Chem 291(40):20869-20875, 2016; US 2021/0047669; U.S. Pat. Nos. 5,409,811; 8,133,672; 8,658,365; and 9,279,155, all of which are herein incorporated by reference. The metal cofactors may be provided in the forms of salts such as MgCl₂ or CoCl₂. In some embodiments herein, the presence/absence or the amount(s) and/or concentration(s) of particular divalent cation(s) can be used to alter the kinetics of polymerases including the rate of nucleotide incorporation.

In any of the embodiments herein, the method can further comprise controlling the presence/absence or the amount(s) and/or concentration(s) of one or more di-cations in the reaction volume. In some embodiments, the one or more di-cations can comprise Ca²⁺, Mg²⁺, Co²⁺, and/or Mn²⁺. In any of the embodiments herein, the method can further comprise controlling the presence/absence or the amount(s) and/or concentration(s) of a di-cation that is not a cofactor of the polymerase, and the di-cation may be Ca²⁺. In some embodiments, Ca²⁺ can stabilize the polymerase without activating its polymerase activity and/or exonuclease activity. In any of the embodiments herein, the method can further comprise controlling the presence/absence or the amount(s) and/or concentration(s) of one or more co-factors of the polymerase in the reaction volume. In any of the embodiments herein, the method can further comprise controlling the presence/absence or the amount(s) and/or concentration(s) of a di-cation that is a cofactor of the polymerase, and the di-cation may comprise Mg²⁺, Co²⁺, and/or Mn²⁺. In any of the embodiments herein, the method can further comprise controlling the presence/absence or the amount(s) and/or concentration(s) of one or more chelating agents, such as EDTA, EGTA, BAPTA, DTPA, or a combination thereof. In some embodiments, the one or more chelating agents can chelate one or more metal ions, such as Co²⁺, Ca²⁺, Mn²⁺, Zn²⁺ and/or Mg²⁺, thereby sequestering these metal ions from polymerases. As such, the presence/absence or the amount(s) and/or concentration(s) of the one or more chelating agents can be adjusted as needed during SBS in order to alter the kinetics of polymerases including their rate of nucleotide incorporation.

In any of the embodiments herein, the method can further comprise controlling a polymerase activity and/or an exonuclease activity of the polymerase, for example, using any combination of the approaches disclosed herein.

In any of the embodiments herein, the detecting step can comprise signal detection in a plurality of detection windows, and there can be intervals between adjacent detection windows. In some embodiments, exposure times (e.g., detection windows) can be selected such that sufficient emission is captured to identify a spot location, for instance when a imaging sensor is used rather than a on-chip or otherwise arrayed system. In any of the embodiments herein, a detection window (e.g., exposure time) can be between about 50 milliseconds (ms) and about 3 second (s), such as between about 50 ms and about 100 ms, between about 100 ms and about 200 ms, between about 200 ms and about 300 ms, between about 300 ms and about 400 ms, between about 400 ms and about 500 ms, between about 500 ms and about 600 ms, between about 600 ms and about 700 ms, between about 700 ms and about 800 ms, between about 800 ms and about 900 ms, or between about 900 ms and about 1 s. In any of the embodiments herein, a detection window (e.g., exposure time) can be about 500 ms. In any of the embodiments herein, a detection window (e.g., exposure time) can be between about 500 ms and about 1 s, between about 1 s and about 1.5 s, between about 1.5 s and about 2 s, or between about 2 s and about 3 s, or more than about 3 s.

In any of the embodiments herein, the internal between detection windows can be minimal such that detection can be viewed as continuous. In any of the embodiments herein, an interval between two adjacent detection windows can be less than about 1 ms, less than about 5 ms, less than about 10 ms, less than about 20 ms, less than about 30 ms, less than about 40 ms, or less than about 50 ms. In any of the embodiments herein, the plurality of detection windows can be uniform in duration or can comprise detection windows of varying durations. Likewise, in any of the embodiments herein, the intervals between adjacent detection windows can be uniform in duration or can comprise intervals of varying durations.

In any of the embodiments herein, the polymerase may catalyze no more than three incorporation events in a detection window. In any of the embodiments herein, the polymerase may catalyze no more than two incorporation events in a detection window. In any of the embodiments herein, the polymerase may catalyze no more than one incorporation event in a detection window.

In any of the embodiments herein, the deactivating step may comprise photobleaching a detectable label, photolysis of the detectable label, photocleavage of a photocleavable linker linking the detectable label, temperature-based detectable label deactivation, pH-based detectable label deactivation, or any combination thereof.

In any of the embodiments herein, the detectable label of a particular incorporated detectably labeled nucleotide can be deactivated (e.g., by photobleaching) during or after the incorporation of one or more subsequent detectably labeled nucleotides. In some embodiments, nucleotide incorporation and photobleaching events are matched. For instance, the fluorophore(s) on each nucleotide may only bleach once and do not recover. In some embodiments, an increase in signal intensity due to incorporation of a nucleotide can be matched with a decrease in signal intensity due to photobleaching of the incorporated nucleotide. In some cases wherein there is a reduction in signal intensity due to photobleaching without a matching increase in signal intensity due to nucleotide incorporation, there may be a failure to register the incorporation or two incorporations have occurred in the same detection window, and one or more of these errors can be resolved using methods disclosed herein, such as HMM-based methods.

In any of the embodiments herein, the deactivation of the detectable labels at the location can be achieved by using photobleaching, an electric field, heat, and/or a change in pH. In any of the embodiments herein, the deactivation can comprise using photobleaching, an electric field, heat, a change in pH or any combination thereof that is local to a surface of the substrate. In any of the embodiments herein, the deactivation can be confined to within about 50 nm, about 100 nm, about 150 nm, or about 200 nm from the surface of the substrate. In any of the embodiments herein, the detectable label(s) of the incorporated detectably labeled nucleotides can be local to the surface of the substrate and deactivated, whereas detectable labels of detectably labeled nucleotides that are not incorporated and not local to the surface of the substrate remain active or capable of being activated.

In any of the embodiments herein, the deactivation of the detectable labels at the location can be stochastic and can occur with a fixed probability at each time point of the time trace.

In any of the embodiments herein, the method can further comprise controlling the rate of the deactivation, such as controlling the rate of photobleaching. In any of the embodiments herein, the rate of photobleaching can be reduced by reducing a laser intensity used for photobleaching and/or reducing the amount or concentration of a free-radical scavenger, such as an oxygen scavenger. In any of the embodiments herein, the free-radical scavenger can comprise 2-mercaptoethanol, dithiothreitol (DTT), tris(2-carboxyethyl)phosphine (TCEP), Na₂SO₃, glucose-coupled glucose oxidase/catalase (GODCAT), or protocatechuate-dioxygenase (PCD), or any combination thereof.

In any of the embodiments herein, the method can further comprise controlling the distance of the deactivation from a surface of the substrate. In any of the embodiments herein, the method can comprise using an evanescent light, an electric field, heat, a change in pH or any combination thereof that is local to the surface of the substrate. In any of the embodiments herein, the distance of the deactivation from a surface of the substrate can be within about 50 nm, about 100 nm, about 150 nm, or about 200 nm from the surface.

In any of the embodiments herein, the signal deactivation rate can be controlled, e.g., by increasing the rate of photobleaching, in order to keep total emission under a threshold total value. In some aspects, it's desirable to tune the deactivation rate to keep the total emission from a strand being sequenced below a threshold total value so as to avoid saturation of a sensor. In some aspects, the rate of signal deactivation is related to the detection window length. In some embodiments, it is desirable to increase the rate of photobleaching to keep the overall total emission per detection window (exposure) within a threshold, and the exposure time and the photobleaching rate may be fine-tuned as needed.

In any of the embodiments herein, the method can comprise limiting the total emission brightness/intensity such that it is generally below a certain threshold, e.g., so as to not exceed a sensor well depth. In some embodiments, the method can further comprises tuning the label/signal deactivation rate so that as to limit the number of simultaneously emitting labels (e.g., fluorophores), since in some cases label-label interactions may become more significant beyond two or three emitting labels (e.g., fluorophores). In some embodiments, by tuning the label/signal deactivation rate, multiple label-label (e.g., dye-dye) interactions and associated non-linearity in emission intensity can be reduced or avoided.

In any of the embodiments herein, the detectable label of the incorporated first detectably labeled nucleotide can be deactivated during or after the incorporation of the second, third, and/or fourth detectably labeled nucleotide; the detectable label of the incorporated second detectably labeled nucleotide can be deactivated during or after the incorporation of the third and/or fourth detectably labeled nucleotide; and/or the detectable label of the incorporated third detectably labeled nucleotide can be deactivated during or after the incorporation of the fourth detectably labeled nucleotide.

In any of the embodiments herein, the detectable labels of incorporated detectably labeled nucleotides using a particular nucleic acid molecules as template can be independently deactivated in any order.

In any of the embodiments herein, the detectable label of a particular incorporated detectably labeled nucleotide can be deactivated (e.g., photobleached) once, resulting in a characteristic decrease in signal intensity at the location of the particular incorporated detectably labeled nucleotide. In any of the embodiments herein, the detectable label of a particular incorporated detectably labeled nucleotide can be deactivated (e.g., photobleached) and its signal intensity does not recover.

In any of the embodiments herein, the deactivating step and/or the detecting step can be carried out as detectably labeled nucleotides are continuously provided to contact the nucleic acid molecule and/or the primer. In some embodiments, the detecting step is performed in real time as the nucleotide incorporation and signal deactivation (e.g., photobleaching) events occur. In some embodiments, the detecting step is not carried out using multiple switchable optical filters each for detecting a different detectable label. In any of the embodiments herein, the detecting step can be carried out using a dichroic filter to split optical signals into channels for detecting a different detectable label in each channel. In any of the embodiments herein, the detecting step can be carried out using total internal reflection fluorescence (TIRF) microscopy. In any of the embodiments herein, the signals in the detecting step can be compensated for background signal.

In any of the embodiments herein, nucleotide identification using the time trace can comprise probabilistically identifying the first, second, third, and/or fourth detectably labeled nucleotides. In any of the embodiments herein, the probabilistically identifying step can comprise assigning a state of signal intensity to each detectable label and decoding the time trace. In some embodiments, the state of signal intensity corresponds to a fixed value of signal intensity (e.g., sum of relative fluorescence over a range of excitation wavelengths). In some embodiments, the state of signal intensity corresponds to a range of signal intensities. In some embodiments, the state of signal intensity corresponds to a log-normal distribution of signal intensities. In some embodiments, the state of signal intensity corresponds to a Gaussian distribution of signal intensities. Methods that use single-molecule intensity distributions to deconvolve fluorescent signals are described, for example, in Mutch et al., “Deconvolving Single-Molecule Intensity Distributions for Quantitative Microscopy Measurements,” Biophysical J. 92(8):2926-2943 (2007), which is incorporated herein by reference in its entirety.

In any of the embodiments herein, decoding the time trace may comprise pairing an incorporation event with a deactivation event of the detectable label of the nucleotide incorporated in the incorporation event. In any of the embodiments herein, decoding the time trace may comprise using a transition probability between two states of signal intensity, and the transition may comprise an incorporation event, a deactivation event (e.g., photobleaching), or an incorporation event and a deactivation event of the same label or different labels at a substrate location. In some embodiments, the transition probability between two states of signal intensity is fixed. In some embodiments, the transition probability between two states of signal intensity is fitted.

In any of the embodiments herein, a Hidden Markov Model (HMM) or the like can be used to analyze the incorporation event(s) and/or the deactivation event(s) at one or more substrate locations by observing states of signal intensity and transitions between the states. In some embodiments, using the HMM comprises providing transition probabilities between states of signal intensity due to nucleotide incorporations and label bleaching where individual label bleaching is not expected to recover. For instance, the HMM can model a first state with two currently unbleached labels emitting, one on the incorporated first detectably labeled nucleotide and the other on the incorporated second detectably labeled nucleotide. In this example, the first state may transition into a second state where the label on the incorporated first detectably labeled nucleotide is bleached, or into a third state where the label on the incorporated second detectably labeled nucleotide is bleached. The first state may also transition into a fourth state due to incorporation of a third detectably labeled nucleotide, while the labels on the incorporated first and second detectably labeled nucleotides are not bleached. In any of the embodiments herein, decoding the time trace may comprise using the Viterbi algorithm for the HMM that represents incorporation and deactivation events. In some embodiments, HMM or a similar model can further be extending to include one or more signal artifacts, e.g., self-quenching, blinking, photo-switching, and/or dye recovery. In some embodiments, nucleotide incorporation, photobleaching, and/or one or more signal artifacts (e.g., self-quenching, blinking, photo-switching, and/or dye recovery) can be modeled during the basecalling process, for instance using HMM or a similar model. HMMs for DNA sequencing have been described, for example, by Boufounos et al., “Basecalling Using Hidden Markov Models,” Journal of the Franklin Institute 341(1):23-36 (2004); Liang et al., “Bayesian Basecalling for DNA Sequence Analysis Using Hidden Markov Models,” IEEE/ACM Transactions on Computational Biology and Bioinformatics 4(3): 430-440 (2007); and Timp et al., “DNA Base-Calling from a Nanopore Using a Viterbi Algorithm,” Biophys J. 102(10): L37-L39 (2012).

In any of the embodiments herein, the determined sequence of the nucleic acid molecule may be no more than 100, no more than 90, no more than 80, no more than 70, no more than 60, no more than 50, no more than 40, no more than 30, no more than 20, no more than 15, or no more than 10 nucleotides in length. In any of the embodiments herein, the determined sequence of the nucleic acid molecule may be about 8, about 12, about 16, about 20, about 24, about 28, about 32, about 36, or about 40 nucleotides in length. In any of the embodiments herein, the determined sequence of the nucleic acid molecule may be between about 5 and about 50 nucleotides in length, such as between about 10 and about 35 nucleotides in length, or between about 15 and about 30 nucleotides in length.

In any of the embodiments herein, the nucleic acid molecule can be a genomic DNA, an mRNA, or a cDNA. In any of the embodiments herein, the nucleic acid molecule can be isolated or derived from a virus, a bacterium, or a fungus. In any of the embodiments herein, the nucleic acid molecule can be a viral DNA or RNA. In any of the embodiments herein, the virus can be a coronavirus, such as a SARS-CoV-2.

Also disclosed herein is a device for determining a sequence of a nucleic acid molecule, comprising a reagent chamber configured to provide a polymerase, a first detectably labeled nucleotide, and/or a second detectably labeled nucleotide to an imaging area. In some embodiments, the device further comprises the imaging area, which may comprise a substrate, wherein a nucleic acid molecule is hybridized to a primer, and the nucleic acid molecule and/or the primer can be immobilized at a location of the substrate. In any of the embodiments herein, the first detectably labeled nucleotide can be complementary to a first nucleotide of the nucleic acid molecule and configured to incorporate into the primer by the polymerase, thereby generating an extended primer comprising the incorporated first detectably labeled nucleotide at the location. In any of the embodiments herein, the second detectably labeled nucleotide can be complementary to a second nucleotide of the nucleic acid molecule and configured to incorporate the extended primer by the polymerase, thereby generating a further extended primer comprising the incorporated first and second detectably labeled nucleotides at the location. In any of the embodiments herein, the device can further comprise a light source configured to provide light for illuminating one or more of the detectable labels.

In any of the embodiments herein, the device can optionally comprise a signal deactivation module configured to deactivate labels such as fluorescent labels. In some embodiments, deactivating the label comprises using conditions local to the surface of the substrate to deactivate the label. In some embodiments, a condition local to the surface can be within about 50 nm, about 75 nm, about 100 nm, about 125 nm, about 150 nm, about 175 nm, or about 200 nm of the surface. In some embodiments, the signal deactivation may be achieved using a light, such as a laser that excites and/or bleaches fluorophores (e.g., via photobleaching).

In any of the embodiments herein, the device can optionally comprise a photobleaching module configured to photobleach the detectable label(s) of the incorporated first and/or second detectably labeled nucleotides. In any of the embodiments herein, the light for illuminating one or more of the detectable labels (e.g., an excitation laser) may function as a bleaching light (e.g., bleaching laser) and can be used to photobleach the one or more detectable labels or other detectable labels. In some embodiments, the excitation laser and the bleaching laser may be the same or different lasers. In some embodiments, the bleaching laser field can be an evanescent light field. In some embodiments, an evanescent light field can be created by total internal reflection of a light beam at an angle larger than the critical angle. In some embodiments, an evanescent field of a laser can be provided through TIRF illumination. In some embodiments, the bleaching laser field can be confined near the surface of the substrate (e.g., within about 50 nm, about 75 nm, about 100 nm, about 125 nm, about 150 nm, about 175 nm, or about 200 nm of the surface) such that energy is spatially concentrated in the vicinity of the substrate. In examples where the excitation laser and the bleaching laser are different, the bleaching laser field can be confined to the surface of the substrate such that nucleotides that are free in solution are not bleached.

In some embodiments, the signal deactivation may be achieved using one or more methods other than photobleaching. In some embodiments, a method that does not depend on photobleaching can be used to create a local environment which stochastically deactivates a label. For example, an electric field can be used to induce a change in pH near the surface and promote dissociation and/or deactivation of a label. See, e.g., May and Hillier, “Rapid and Reversible Generation of a Microscale pH Gradient Using Surface Electric Fields,” Analytical Chemistry 77: 6487-6493 (2005), incorporated herein by reference in its entirety. In some embodiments, a label can be tethered to an oligonucleotide, where local pH causes dissociation and removal of the label. In some embodiments, the label can be covalently linked (e.g., directly via a covalent bond or indirectly via a linker) to the oligonucleotide, and a local pH change can cause cleavage of the covalent bond or linker, thereby releasing the label. In some embodiments, the label can be noncovalently linked (e.g., via nucleic acid hybridization or other hydrogen bond and/or van der Waals contacts) to the oligonucleotide, and a local pH change can cause melting of the duplex or complex, thereby releasing the label. In some embodiments, local heating and/or an electric field itself may be used to promote label deactivation. In some embodiments, a method disclosed herein comprises only deactivating those labels near the surface of the substrate, e.g., within about 50 nm, about 75 nm, about 100 nm, about 125 nm, about 150 nm, about 175 nm, or about 200 nm of the surface, thereby promoting deactivation of labels on incorporated nucleotides, while minimizing and/or preventing deactivation of labels on nucleotides that are in solution and not yet incorporated. In some embodiments, only labels on incorporated nucleotides are deactivated during a signal deactivation step and labels on free nucleotides in solution are not deactivated in the signal deactivation step, thus preventing the free nucleotides from being incorporated in a deactivated state.

In any of the embodiments herein, the device can further comprise a detection module configured to detect signals or absence thereof associated with the detectable labels at the location over time, thereby generating a time trace of signal intensity associated with the incorporation and/or photobleaching of detectable labels of detectably labeled nucleotides at the location.

In any of the embodiments herein, the reagent chamber and the imaging area can be connected by a fluidic communication configured to continuously provide the polymerase, the first detectably labeled nucleotide, the second detectably labeled nucleotide, and/or one or more other reagents to the imaging area. In any of the embodiments herein, the device may but does not need to comprise a flow cell outlet configured to remove molecules of one or more of the polymerase, the first detectably labeled nucleotide, the second detectably labeled nucleotide, and/or one or more other reagents from the imaging area; however, the device may comprise one or more vents. In any of the embodiments herein, the device may be configured for single use. In any of the embodiments herein, the reagent chamber and/or the imaging area can be configured for single use, whereas the light source, photobleaching module, and/or the detection module can be reused one or more times.

Also described herein is a system, comprising one or more processors and a non-transitory storage medium comprising one or more programs executable by the one or more processors to receive information related to one or more sequencing reads generated using a method disclosed herein and/or perform any one or more of the methods disclosed herein.

### BRIEF DESCRIPTION OF THE DRAWINGS

The drawings illustrate some embodiments of the features and advantages of this disclosure. These embodiments are not intended to limit the scope of the appended claims in any manner.

FIGS. 1A-1B show results of a simulation of 0.1% phasing and prephasing.

FIG. 2 shows an exemplary depletion or decimation process in a cluster, where deactivated strands are marked solid black.

FIG. 3 shows polymerase errors can be propagated during clonal amplification to generate a cluster and the spatial relationships among strands in the cluster can be used to infer base call quality.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

The present disclosure in some aspects relates to methods and systems for determining the nucleotide sequence of individual nucleic acid molecules using optical techniques, referred to herein as “single molecule optical sequencing.” In some embodiments, disclosed herein are methods for imaging labeled nucleotides added onto a nucleic acid molecule mounted on a substrate, e.g., a solid surface, wherein the nucleic acid molecules is sequenced using sequencing-by-synthesis (SBS). Any one or more of the labeled nucleotides can be labeled with only one kind of label (e.g., a fluorophore appearing as “red” or “green”), and may be labeled with one or more molecules of the same label. Further, any one or more of the labeled nucleotides can be labeled with two or more kinds of labels (e.g., a “red” first fluorophore and a “green” second fluorophore such that the labeled nucleotide appears as “yellow”), and may be labeled with one or more molecules of each kind of label. For a multiply labeled nucleotide, the ratio of different kinds of labels can be tuned as needed, e.g., such that labeled nucleotides having different ratios of distinct labels may be distinguished.

The DNA sequencing method most commonly used until the 2000s was dideoxy chain terminator sequencing (Sanger et al., PNAS 74(12): 5463-5467 (1977), incorporated herein by reference in its entirety for all purposes). However, this method is time-consuming, labor-intensive, expensive, and low throughput. To overcome some of these deficiencies massively parallel sequencing (MPS) approaches were developed by Solexa and others (e.g., U.S. Pat. No. 7,115,400 incorporated herein by reference in its entirety for all purposes). These approaches sequence large numbers of nucleic acids in parallel, and have drastically reduced cost-per-base.

However, key deficiencies remain. In particular, while MPS has reduced the cost per base, the cost per run remains high (>$400). Common MPS approaches also work using clusters of amplified DNA. As such they are limited to DNA sequencing, and cannot be used to directly sequence RNA.

Single molecule sequencing (e.g., as implemented by Pacific Biosciences, Helicos and others) addresses some of these issues. However, these approaches have not resulted in lower run cost. In single molecule SBS, photobleaching has been proposed as a method of deactivating labeled nucleotides to avoid signal accumulation (Braslavsky et al.). The counting of discrete bleaching events has been proposed as a method of resolving multiple incorporations (e.g., U.S. Pat. No. 6,221,592 incorporated herein by reference in its entirety for all purposes). In these methods, incorporated dyes are bleached to prevent signal accumulation, since residual signals from previous cycles would interfere with detection in later cycles. Photobleaching must be taken to completion to remove all dye labels before labeled nucleotides are added to start a new cycle.

By bleaching to completion, the existing SBS methods can only be used in a cyclic sequencing context. In addition to this, bleaching to completion exposes the nucleic acid strand to light which may case photo-damage to the nucleic acid before sequencing is complete. It is therefore desirable to minimize illumination of the strands being sequenced. In addition to this, it is not obvious how photo-bleaching would be used in a real-time sequencing context, where nucleotides are continuously and not cyclically introduced.

The present disclosure in some aspects relates to single molecule nucleic acid sequencing methods where dye deactivation (for example by photobleaching) limits signal accumulation but is not generally taken to completion prior to incorporation of additional labeled nucleotides in a given strand being sequenced. In some embodiments, a drop in signal intensity (e.g., emission) resulting from dye deactivation may be used to infer information about the strand under synthesis (and the complementary template strand), as part of a nucleic acid sequencing approach. It should be noted that photobleaching and/or any other suitable method of dye deactivation may be used. Exemplary photobleaching techniques are described, e.g., in Chen et al., Mol Biol Cell, 25(22): 3630-42 (2014), incorporated herein by reference in its entirety for all purposes.

In some embodiments, provided herein is a single molecule sequencing-by-synthesis method where nucleic acid strands are attached to a solid surface and then extended by a polymerase (e.g., by a DNA polymerase or a reverse transcriptase) to incorporate a nucleic acid molecule (e.g., a nucleotide) comprising a fluorescent (or otherwise emitting) label to the 3′ terminus of a sequencing primer hybridized to a nucleic acid strand.

In some embodiments, an imaging platform capable of resolving single dyes at multiple locations on a substrate is used to image the dyes, and determine the “intensity” of a nucleic acid “spot.” In some embodiments, the term “intensity” used herein refers to a value computed from dye emissions of a single nucleic acid imaged as a “spot.” In some embodiments, the intensity may comprise emissions from one or more molecules of the same dye or different dyes, and may be corrected, for example to compensate for background signal such as background illumination (e.g., background fluorescence, such as autofluoresence). In some embodiments, the imaging system can be used to determine when labels are incorporated (which results in increases in intensity), and when bleaching events have occurred (which results in decreases in intensity).

In some embodiments, by algorithmically combining information regarding incorporation events (intensity increases) and photobleaching events (intensity decreases), the sequence of a single nucleic acid strand can be probabilistically determined. Such an approach is simpler than current sequencing approaches which require multiple reagent cycles, and does not require a nano-fabricated surface.

In some embodiments, disclosed herein are methods where photobleaching is not taken to completion during a single incorporation/imaging cycle. In some embodiments, stepwise increases in signal intensity are used to register the incorporation of labeled nucleotides. In some embodiments, photobleaching steps are used to provide information to determine not just the number of incorporations, but the nucleotide sequence of a strand under synthesis. In some embodiments, multiple labels can be used, where the labeled nucleotides can be distinguished from one another based on the type and/or number of label(s) on an individual labeled nucleotide. These labels may emit at a specific wavelengths, or when filtered, produce a characteristic increase in signal intensity. In some embodiments, a nucleotide incorporation event and a signal deactivation event of the incorporated nucleotide can be matched or paired. For instance, a label that produces a characteristic increase in signal intensity can result in a corresponding characteristic decrease in signal intensity when the label is bleached. In some embodiments, between two time points or by comparing signal intensities between two detection time windows, a change in registered intensity may reflect the type of labeled nucleotide incorporated and be used to determine the complementary sequence in the strand being sequenced.

In some embodiments, labeled nucleotides may but do not need to be added cyclically. In some embodiments, a method disclosed herein may comprise one or more cycles in which one or more labeled nucleotides are added, signals associated with nucleotide incorporations are detected, signals of the incorporated nucleotides are deactivated, and the substrate is washed to remove labeled nucleotides and optionally cleaved labels, before additional labeled nucleotides are added to sequence the next base. In cases where one or more labeled nucleotides are added cyclically, a single label may be used to label the one or more labeled nucleotides in a cycle, for example, similar to a 2-channel SBS chemistry using “red” for C, “green” for T, “red” and “green” appearing as “yellow” for A, and unlabeled for G. In some embodiments, a method disclosed herein may comprise using a single label and introducing labeled nucleotides in one or more cycles, where in each cycle or flow only labeled nucleotides comprising one nucleotide type (e.g., A, T, C, or G) and the single label are introduced in the sequencing reaction, and nucleotide incorporation/non-incorporation is monitored in the one or more cycles. In these embodiments, nucleotides introduced in one cycle are either signal-deactivated (if incorporated) or removed (if not incorporated) before nucleotides of the same type or different types and labeled with the same single label are introduced.

In some embodiments, a method disclosed herein is a single molecule sequencing method, for instance, for DNA or direct RNA sequencing. In some embodiments, the method can use a single detection channel, e.g., for detecting signal intensity of a plurality of different labels. For example, a single channel is sufficient to detect and distinguish signals associated with two fluorophores, ATTO 532 and ATTO 542, based on their characteristic intensity (e.g., sum of relative fluorescence over a range of wavelengths). In some embodiments, the method is a single molecule and single channel sequencing method. In some embodiments, the method is unterminated and/or non-cyclical. For instance, the method does not require the use of chain terminators (e.g., a reversible terminator that can terminate primer extension reversibly) or sequencing cycles comprising signal deactivation and/or label removal. In some embodiments, the method utilizes labeled nucleotides but the labels do not need to cleaved and/or removed from incorporated nucleotides.

In some embodiments, labeled nucleotides may be added and imaged during incorporation in a real-time sequencing method. A marked spot can be created from the point spread function (PSF) of a single or emitter or group of diffraction limited emitters, for example multiple labels on a single nucleic acid strand. Images may be registered and segmented to identify spot locations. Once a spot is identified, background signal (e.g., due to background fluorescence and/or autofluoresence) may be calculated and removed from images of the spot. Other signal artifacts (for example foreground illumination variation) may be compensated for. A characteristic signal for each spot may be extracted. A number of methods may be used for extracting signals. For example, a characteristic signal may be obtained by extracting the peak value within a spot, and/or by fitting a point spread function (for example a 2D Gaussian function) to the spot profile and using the peak value or other features from the fit. In some embodiments, this characteristic value is termed the “intensity” or “signal intensity” which are used interchangeably herein.

In some embodiments, the intensity of a spot may be extracted over a number of frames to produce an intensity profile (e.g., in the form of a time trace) for a spot. In some embodiments, the intensity profile (e.g., time trace of signal intensity) of a spot is generated from labeled nucleotides incorporated into a strand under synthesis. This profile maybe further corrected and processed to determine a nucleic acid sequence of the complementary template nucleic acid which can be RNA or DNA.

In some embodiments, labeled nucleotides are incorporated into a strand under synthesis (for example using a polymerase or reverse transcriptase). In some embodiments, labeled nucleotides once incorporated do not need to be photobleached before one or more subsequent labeled nucleotides are incorporated. In some examples, first a single nucleic acid strand (5′-AATAG-3′) is attached to a surface and a first labeled nucleotide (“A” in this example) is incorporated using a polymerase or reverse transcriptase. A second labeled nucleotide (“T” in this example) may be present in the sequencing reaction before, during, and/or after the first labeled “A” nucleotide is incorporated. Then, the second labeled “T” nucleotide can be incorporated before the first labeled “A” nucleotide is photobleached. Then, a third labeled nucleotide (“T” in this example) can be incorporated after the first labeled “A” nucleotide is photobleached but before the second labeled “T” nucleotide is photobleached. The third labeled “T” nucleotide can be bleached while the second labeled “T” nucleotide is not yet photobleached. The second labeled “T” nucleotide can then be photobleached. A time trace of the detected signals at the spot can be generated and used to determine a sequence of the nucleic acid strand, e.g., 5′-AAT-3′ which is complementary to the synthesized 5′-ATT-3′ sequence in the sequencing primer strand.

In some embodiments, labeled nucleotides may be incorporated an imaged under illumination (for example objective or prism style TIRF illumination). In some embodiments, labeled nucleotides may be incorporated and photobleaching of the incorporated labeled nucleotides occur stochastically. In some embodiments, nucleotides comprising different bases may be labeled with the same label. In some embodiments, nucleotides comprising different bases may be labeled using labels having different excitation wavelengths and/or different emission wavelengths. In some embodiments, nucleotides comprising different bases may be labeled using labels which result in differing intensity at a given wavelength or across a given range of wavelengths.

In some embodiments, photobleaching and/or any suitable method of dye deactivation may be used. For example, a photocleavable fluorescent nucleotide may be used, for instance, as described in Meng et al., “Design and Synthesis of a Photocleavable Fluorescent Nucleotide 3′-O-Allyl-dGTP-PC-Bodipy-FL-510 as a Reversible Terminator for DNA Sequencing by Synthesis,” J. Org. Chem. 71, 8, 3248-3252 (2006), incorporated herein by reference in its entirety for all purposes. Other methods of dye deactivation based on temperature or pH may also be used.

Photobleachable nucleotides may include 5-(3-Aminoallyl)-2′-deoxyuridine-5′-triphosphate, labeled with ATTO 532, Triethylammonium salt (Jena Biosciences, Germany) or similar ATTO labeled nucleotides. Nucleotides may be introduced at a concentration appropriate to the experimental conditions, for example, 10 nM, 20 nM, 30 nM, 40 nM, 50 nM, 60 nM, 70 nM, 80 nM, 90 nM, or 100 nM, or in a range between any of the aforementioned values. Nucleotides may be constructed where photodamage is used to cause dye cleavage. Nucleotides may also be constructed to contain multiple emitters, providing differing emission strength. Such nucleotides may contain a cleavable element such that all emitters will be simultaneously removed/deactivated.

Nucleotides may be incorporated using a suitable polymerase, for example a 9° N or related polymerase, or Klenow fragment, or the SuperScript® III reverse transcriptase (Invitrogen) or another reverse transcriptase.

In some embodiments, nucleotides are labelled with labels which result in differing intensity. A trace may be extracted from acquired images where nucleotide incorporation and imaging has proceeded as described above using said labels of differing intensity. Such labels result in a convolved signal which photobleaching events occur stochastically. Both incorporation events (increases in intensity) and bleaching events (decreases in intensity) provide information which can aid in determining the nucleotide sequence of a strand under synthesis and the complementary strand being sequenced. Nucleotide labels may be selected such that labels show differing emission levels over the same range of wavelengths. For example ATTO 532 and ATTO 542 may be used which at 537 nm show relative emission levels of 0.443 and 0.104, respectively.

In some embodiments, a method disclosed herein comprises controlling the photobleaching rate, such as by using a free-radical scavenger, for example β-mercaptoethanol (Yanagida et al., 1986, in Applications of Fluorescence in the Biomedical Sciences, Taylor et al. (eds) Adaln R. Liss Inc., New York, pp. 321) or glucose oxidase. For example, in some embodiments, the method comprises tuning the photobleaching rate to keep total emission under a threshold total value. In some embodiments, a method disclosed herein comprises preventing emissions saturating the image sensor well depth at a given exposure time.

A time trace of signal intensity may be analyzed and deconvoluted, for example using a Hidden Markov Model (HMM) capable of decoding a di-nucleotide sequence where nucleotides are labeled with varying brightness. The “A” nucleotide can be labelled with an intensity of magnitude 1 and the “T” nucleotide can be labelled with an intensity of magnitude 2 (double the intensity of “A”). Such an HMM using a Viterbi or other decoder can be used to basecall an intensity trace. The transitions in such a model represent the nucleotide type that is incorporated. The states represent intensity levels obtained from an intensity trace as described above. The transitions labeled Pb represent photobleaching events. The HMM can be used to model any combination of 3 nucleotide types illuminated at any one time. To simplify the example, only 2 nucleotide types are shown here (“A” and “T”), however the model may be extended to 4 nucleotides where more than 3 nucleotide types are illuminated at any one time using known methods. Self-transitions are not shown, which would model a steady state. Additional states may be added to compensate for multiple bleaching events in a single sample. In some embodiments, states may be added to model dye self-quenching, blinking, photo-switching, and/or dye recovery. States may model emission intensity as a fixed value, a range, or as a Gaussian distribution. The transition probabilities for incorporations may be fixed (as determined experimentally) or fitted to each experiment. Similarly, the photobleach transition probabilities (Pb) may be fixed (as determined experimentally) or fitted to each experimental dataset.

While the HMM can be demonstrated using two transition types representing adenine (A), thymine (T), it may also be extended with cytosine (C) and guanine (G) nucleotides. The HMM may also represent the sequencing-by-synthesis and photobleaching of a RNA strand.

In some embodiments, a method disclosed herein can be used to provide rapid and inexpensive sequencing solutions, for instance, in response to a pandemic such as COVID-19. Such pandemic scale sequencing methods can rival qPCR based methods in terms of cost, at a cost per run much lower than existing sequencing-by-synthesis methods that rely on flow cell cycles. In some embodiments, the sequencing methods disclosed herein can be used to diagnose a disease or condition, such as viral infection. In some embodiments, the sequencing methods disclosed herein overcome limitations of qPCR based methods and achieve improved detection accuracy.

In some embodiments, provided herein are low-cost sequencing methods (e.g., for pandemic response) that can accurately detect a plurality of nucleic acid molecules, including viral RNA in a biological sample. For instance, the biological sample can be processed to extract viral nucleic acid (e.g., RNA) while optionally depleting human nucleic acid (e.g., RNA). The extracted viral nucleic acid can be sequenced using a method disclosed herein in a massively parallel, high throughput manner. As such, the present/absence, amount, and sequence of viral nucleic acid can be rapidly detected using a method comprising RNA extraction from patient samples and direct RNA sequencing according to some embodiments of the present disclosure. In some embodiments, no reverse transcription of RNA to cDNA is required. In some embodiments, no multiplex PCR of the extracted RNA or cDNA reverse transcribed therefrom is required. In some embodiments, no further processing of the extracted nucleic acid (e.g., RNA) is required prior to sequencing using a method disclosed herein. For instance, in some embodiments, the extracted nucleic acid (e.g., RNA) does not need to be tagmented and/or amplified prior to sequencing. In some embodiments, a method provided herein can be used to sequence at least about 10, 11, 12, 13, 14, 15, 16, 17, 18, 19, 20, 21, 22, 23, 24, 25, 26, 27, 28, 29, or 30 consecutive nucleotides or longer nucleotide sequences, with less than about 10%, less than about 5%, or less than about 1% error rate in between about 100,000 and about 1 million sequencing reads.

### II. Samples and Nucleic Acid Molecules

The nucleic acid molecules used in the methods described herein may be obtained from any suitable biological source, for example a tissue sample, a blood sample, a plasma sample, a saliva sample, a fecal sample, or a urine sample. The polynucleotides may be DNA or RNA molecules. In some embodiments, RNA molecules are reverse transcribed into DNA molecules prior to hybridizing the polynucleotide to a sequencing primer. In some embodiments, RNA molecules are not reverse transcribed and are hybridized to a sequencing primer for direct RNA sequencing. In some embodiments, the nucleic acid molecule is a cell-free DNA (cfDNA), such as a circulating tumor DNA (ctDNA) or a fetal cell-free DNA.

Examples of nucleic acid molecules include DNA molecules such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid molecules also include RNA molecules such as various types of coding and non-coding RNA, including viral RNAs. Examples of the different types of RNA molecules include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the nucleic acid molecules disclosed herein are non-capped mRNA, a non-polyadenylated mRNA, and a non-spliced mRNA. The RNA analyte can be a transcript of another nucleic acid molecule (e.g., DNA or RNA such as viral RNA).

In some embodiments, a nucleic acid molecule may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In some embodiments, a nucleic acid molecule can be extracted from a cell, a virus, or a tissue sample comprising the cell or virus. Processing conditions can be adjusted to extract or release nucleic acid molecules (e.g., RNA) from a cell, a virus, or a tissue sample.

### III. Sequencing Methods

In some embodiments, disclosed herein is a method for nucleic acid sequencing comprising colony surface amplification (e.g., using bridge amplification or an isothermal amplification method). Exemplary colony surface amplification methods include those disclosed in U.S. Pat. Nos. 7,115,400, 7,541,444, 7,771,973, 8,071,739, 8,597,881, 8,652,810, 9,121,060, 9,297,006, 9,388,464, 10,370,652, 10,513,731, and US 2020/0399692, each incorporated herein by reference in its entirety for all purposes.

In some embodiments, an amplified cluster of nucleic acid molecules (e.g., DNA) is created on a surface. In some embodiments, an amplified cluster is clonal and all nucleic acid strands in the cluster comprise at least one identical sequence to be determined, accepting polymerase errors (e.g., if a nucleotide difference is introduced due to polymerase error during clonal amplification, the sequences in two strand can be considered an identical sequence). In some embodiments, an amplified cluster can comprise sequences from one or more concatemers, such as a rolling circle amplification product comprising multiple copies or repeats of a unit sequence, and the copies or repeats comprise at least one identical sequence to be determined and can be cleaved from the rolling circle amplification product.

In some embodiments, a cluster and an identical sequence shared among molecules (or shared by repeats in the same molecule) can be sequenced, e.g., using sequencing-by-synthesis (SBS), sequencing-by-binding (SBB) or sequencing using a dye labeled polymer with multiple, identical nucleotides attached (e.g., avidity sequencing).

In some embodiments, in a method disclosed herein, reversibly terminated nucleotides are incorporated into a strand under synthesis using a polymerase. In some embodiments, the nucleotides are labeled with a cleavable fluorophore, such that each nucleotide type may be specifically detected. Once detected, the label may be removed, and the blocking group (e.g., a terminator) can be removed. In some embodiments, subsequent nucleotides may be incorporated and the complete sequence of the identical sequences in the strands in the cluster is determined.

A cluster based amplification approach generally provides more emitted signals than available with conventional single molecule approaches. Cluster based amplification can provide advantages in terms of improved signal-to-noise (SNR) ratios and allows cheaper and simpler cameras to be used. The approach also means that a certain amount of photo-damage may be tolerated. If a fraction of molecules (strands) within a cluster are photodamaged, the remaining molecules may still provide sufficient signal to allow sequencing to continue and the sequence to be determined. However, a major limitation of cluster based sequencing is phasing.

Phasing is the tendency of molecules to become “out of sync” in a cluster. This may be through the multiple incorporation of nucleotides (e.g., poor blocking) or non-incorporation of a complementary nucleotide. Recent Illumina chemistries have reported phasing on the order of 0.1%. See, e.g., U.S. Pat. No. 11,293,061 and US 2022/0220553, each incorporated herein by reference in its entirety for all purposes. Even at these modest levels, phasing corrections are needed to correct for signals artifacts caused by phasing. This phasing correction process forms part of the base-calling algorithm, which corrects for signal artifacts and forms an estimate of the correct nucleotide. See, e.g., Whiteford et al., “Swift: primary data analysis for the Illumina Solexa sequencing platform,” Bioinformatics, 2009, 25(17): 2194-9, incorporated herein by reference in its entirety for all purposes. However, even at modest levels of phasing, the ability to correct for phasing artifacts is limited and ultimately limits read length. As successive cycles of incorporation proceed, strands become more and more out of phase, smearing the signal across multiple cycles until it is not easily detectable.

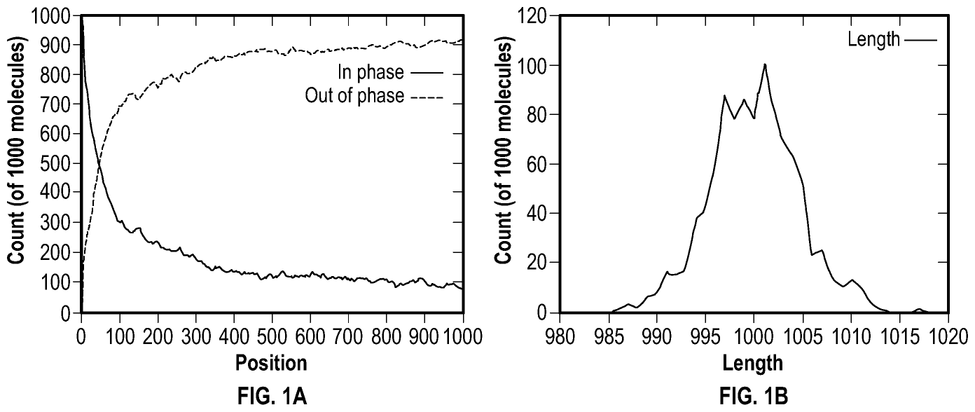

A cluster containing 1,000 strands was simulated. FIG. 1A shows with 0.1% (pre-phasing and phasing) phasing by cycle 1,000, 92.2% of the signals were out of phase. FIG. 11B shows the distribution of sequence lengths at cycle 1000. In this simulation (simulation code shown below), the full width at half maximum (FWHM, the difference between the two values of the independent variable at which the dependent variable is equal to half of its maximum value) was ˜10, suggesting that strands were significantly out of phase. If phasing/pre-phasing (e.g., non-incorporation and multi-incorporation rates) significantly differ, phasing issues can likely be worse as unbalanced phasing will push strands further out of phase. Without continuing the sequencing process indefinitely, it will therefore be impossible to obtain the sequence information necessary to unambiguously determine the strand sequence up to this point.

```
#include <iostream>
#include <vector>
#include <string>
#include <stdlib.h>
using namespace std;
int main( ) {
  vector<string> strands(1000);
  for(int n=1;n<1001;n++) {
    for(int i=0;i<strands( );i++) {
        int r = rand( )%1000;
```

| | |
|---|---|
| if(r<970) strands[i] += ″N″; else | // Normal incorporation (in phase) |
| if(r<990) strands[i] += ″″; else | // No incorporation |
| strands[i] += ″NN″; | // Multiple incorportation |
| } | |
| int inphase=0; | |
| int outphase=0; | |

```
    for(int i=0;i<strands.size( );i++) {
      if(strands[i].size( ) == n) inphase++;
```

else outphase++;

```
    }
    cout <<n>> ″ ″ << inphase << ″ ″ << outphase<< endl;
  }
    vector<int> len(10000);
    for(int i=0;i<strands.size( );i++) {
      len[strands[i].size( )]++;
    }
    cout << ″LENS″ << endl;
    for(int n=0;n<10000;n++) {
      cout << n << ″ ″ << len [n] << endl;
    }
}
```

In some embodiments, a method disclosed herein uses a cluster/colony based approach to obtain information about the original template strand used to form the cluster/colony, where one or more disadvantages of conventional cluster/colony sequencing approaches, such as phasing issues. In some embodiments, a method disclosed herein does not require phasing correction. The advantage of not requiring phasing correction can be seen in circular consensus sequencing methods, such as those disclosed in U.S. Pat. No. 9,910,956 and US 2018/0211003, each incorporated herein by reference in its entirety for all purposes.

In some embodiments, a method disclosed herein comprises clonal amplification of a nucleic acid sequence to be sequenced, e.g., using bridge amplification or an isothermal amplification, which result in a cluster (which can be one or more molecules) containing multiple copies of an original template on a surface. In some embodiments, the method comprises decimating the cluster. In some embodiments, the decimation comprises stochastically or otherwise depleting active sequences (e.g., strands or copies of a sequence which are able to function as a template to incorporate labelled nucleotides) in a cluster. In some embodiments, a mixture of nucleotides can be contacted with the cluster, and the mixture can comprise one or more nucleotides that are not terminated (that is, the nucleotide(s) can be incorporated and allow incorporation of an additional nucleotide to the incorporated residue) and one or more nucleotides that are terminated (that is, the nucleotide(s) can be incorporated but not allow incorporation of an additional nucleotide to the incorporated residue). In some embodiments, the one or more nucleotides that are not terminated can be any one or more of A, T/U, C, and G nucleotides. In some embodiments, the one or more nucleotides that are not terminated can be natural nucleotide(s). In some embodiments, the one or more nucleotides that are terminated can be any one or more of A, T/U, C, and G nucleotides. In some embodiments, the one or more nucleotides that are terminated can comprise irreversibly terminated nucleotides, or terminated nucleotides that are reversible under different conditions. The one or more terminated nucleotides can but do not need to be reversibly terminated. In some embodiments, terminated nucleotides may be similar to those traditionally used for Sanger sequencing (for example, modified dideoxynucleotide triphosphates, lacking a 3′-OH group required for the formation of a phosphodiester bond between two nucleotides).

In some embodiments, for nucleotide molecules containing a particular base (e.g., A, T/U, C, or G), all of the nucleotide molecules of that base contacted with the cluster can be terminated or not terminated, as long as the mixture of nucleotides contacted with the cluster comprises one or more nucleotide molecules that are terminated. In some embodiments, for nucleotide molecules containing a particular base (e.g., A, T/U, C, or G), the nucleotide molecules can comprise one or more molecules that are not terminated, as well as one or more molecules that are terminated. In some embodiments, for nucleotide molecules containing a particular base (e.g., A, T/U, C, or G), the nucleotide molecules can comprise one or more molecules that are reversibly terminated, as well as one or more molecules that are irreversibly terminated.

For instance, a mixture containing all A, T/U, C, and G nucleotides each both with reversible terminators and a fraction (for example, about 5%, about 10%, about 20%, about 25%, about 30%, about 35%, about 40%, about 45%, about 50%, about 55%, about 60%, about 65%, about 70%, about 75%, about 80%, about 85%, about 90%, about 95%, or more) of nucleotides containing non-reversible terminators.

In other examples, nucleotides of each base type can be individually contacted with the clusters, using un-terminated nucleotides and a fraction (for example, about 5%, about 10%, about 20%, about 25%, about 30%, about 35%, about 40%, about 45%, about 50%, about 55%, about 60%, about 65%, about 70%, about 75%, about 80%, about 85%, about 90%, about 95%, or more) of terminated nucleotides.

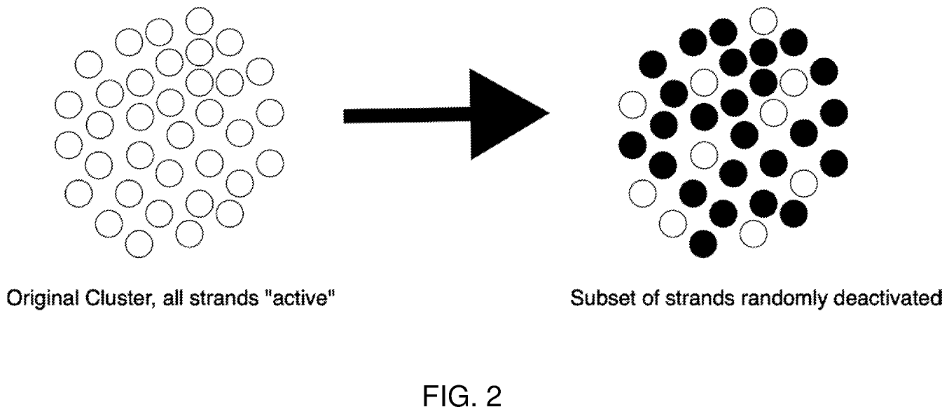

The result of the depletion process can be a cluster where active strands are more “spread out” than would otherwise be the case (see e.g., FIG. 2). This may be desirable during subsequent steps. Clusters may be sequenced using existing nucleotides and sequencing-by-synthesis methods, and the optical system is configured to allow individual strand emissions to be detected, e.g., using single molecule sequencing. As such, each strand within the cluster can be independently sequenced. This process may use reversible terminators and occur cyclically or may proceed through the real time imaging of nucleotides as they are incorporated. In a real time process, fluorescence may be removed by photobleaching or other stochastic methods, for instance, as described in U.S. Provisional Patent Application No. 63/248,951, filed Sep. 27, 2021, entitled “Methods and Compositions for Nucleic Acid Sequencing,” U.S. Provisional Patent Application No. 63/213,795, filed Jun. 23, 2021, entitled “Real-time Sequencing by Photobleaching,” and WO 2022/271701, each incorporated herein by reference in its entirety for all purposes.

Super-resolution approaches may also be used to allow active strands within a cluster to be spaced more closely. For example PAINT based approaches maybe used, where nucleotides contain “blinking” labels, or structured illumination approaches may be used to provide no-diffraction limited localization of nucleotides/strands.

In some embodiments, a sequencing process disclosed herein results in a number of sequences, for instance, one for each strand that is not deactivated. Single molecule sequencing in general results in higher error rates than colony SBS (for example Solexa Illumina style sequencing). As such each read likely contains one or more errors. These errors can be from a number of sources. In some embodiments, an error can be stochastic and result from random effects such as bleaching of dyes preventing their registration. Algorithmically, in some embodiments, information from the multiple strands in a cluster can be combined to correct for these errors. For example, a traditional consensus approach can be used to align strands and generate a consensus sequence.

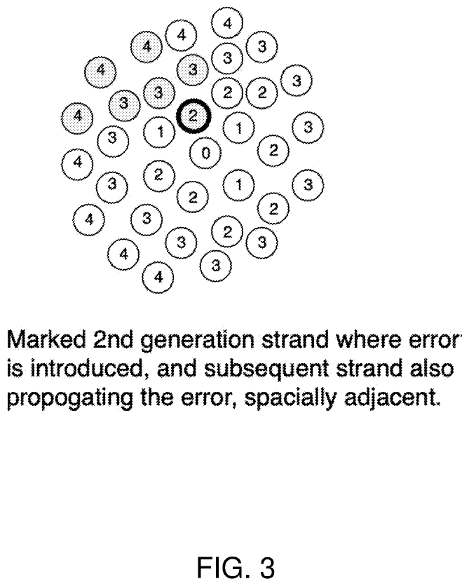

In some embodiments, a method disclosed herein provides a tool for correcting another error type. These are errors which are created as part of the cluster generation process. For instance, each time the polymerase copies an existing strand an error may be introduced. As sequence and location information for all strands can be provided, it can be estimated which strand is the original template or an error-free copy of the original template, and which strands are errored copies each containing one or more errors. FIG. 3 shows such an error, and how its spatial location within the clonal cluster allows identification of such errors. These errors may either be corrected (if sufficient information is available), masked, or used to infer lower base call quality. In the figure, a second generation strand where error has been introduced is marked, and subsequent generations of strands also propagate the error in the second generation strand and are spatially adjacent to the second generation strand.

In some embodiments, a method disclosed herein can comprise further modifications to strands in addition to the decimation. In some embodiments, a method disclosed herein can decouple phasing errors from the additional information provided through the clonal amplification process, and as such strands within a cluster/colony can be further modified to provide additional information.

For example, “phasing” between strands may be increased, such that they are significantly out of sync with each other, thereby providing information about different parts of the strand. In some embodiments, increasing phasing can be achieved by, for example, incorporating a mixture of natural and reversibly terminated nucleotides. These will incorporate and terminate (reversibly) stochastically, pulling strands out of phase (sync). In some embodiments, increasing phasing can be achieved by other methods, for example hybridization of random primers, such that the sequencing-by-synthesis process can be started at different points on the strand.

In some embodiments, by increasing phasing, shorter reads starting from different points on a strand can be used to generate synthetic long reads. By single molecule sequencing each strand, sequence information covering different regions of the original template can be obtained. These sequences maybe combined via multiple alignment to generate a synthetic long read. Such long reads have utility in many applications (for example de novo sequencing, repeat counting, detection of structural variation etc.). Moreover, such a process does not have the disadvantages of other synthetic long read approaches (for example those based on introducing mutation to fingerprint a strand), for example, approaches where errors are intentionally introduced and such errors will need to be resolved through overlapping multiple strands, a process which itself may introduce bias among other issues. Thus, in some embodiments, a method disclosed herein does not comprise intentionally introducing one or more mutations in a strand.

While the discussion above references cluster colony approaches. This approach is also applicable using similar techniques to both nanoball and bead based amplification approaches. It's also noted that other methods may be used to create “depleted” structures. For example, methods similar to those used in expansion super-resolution microscopy. Or, for example melting can be used to melt an initial double stranded template (or synthesized double stranded template) which may re-associate near the original template (possibly under electrostatic or other force). This may aid in the creation of a cluster/amplified structure where templates as sufficiently spreadout to allow the sequencing of individual molecules. The surface may also be patterned with oligos rather than homogeneously covered at high density. This may be through turning the density of oligo adapters such that bridge amplified molecules are likely to hybridize to an oligo at sufficient distance. Or oligos may be nanopatterned on the surface to assist in the “spreading out” of templates to allow the above.

In certain sequencing-by-synthesis methods, a first population of detectably labeled nucleotides (e.g., dNTP) are introduced into a reaction chamber to contact a template nucleotide hybridized to a sequencing primer in the chamber, and a first detectably labeled nucleotide (e.g., A, T, C, or G nucleotide) is incorporated by a polymerase to extend the sequencing primer in the 5′ to 3′ direction using a complementary nucleotide (a first nucleotide residue) in the template nucleotide as template. A signal from the first detectably labeled nucleotide can then be detected. The first population of nucleotides may be continuously introduced into the reaction chamber (e.g., a flow cell), but in order for a second detectably labeled nucleotide to incorporate into the extended sequencing primer, nucleotides in the first population of nucleotides that have not incorporated into a sequencing primer generally must be removed (e.g., by washing), and a second population of detectably labeled nucleotides must be introduced into the chamber. Then, a second detectably labeled nucleotide (e.g., A, T, C, or G nucleotide) is incorporated by the same or a different polymerase to extend the already extended sequencing primer in the 5′ to 3′ direction using a complementary nucleotide (a second nucleotide residue) in the template nucleotide as template. Thus, in these methods, cycles of introducing and removing detectably labeled nucleotides must be performed.

In contrast, in one embodiment of a method disclosed herein, the first detectably labeled nucleotide and the second detectably labeled nucleotide do not need to be introduced into the chamber in separate cycles. In some embodiments, the second detectably labeled nucleotide is already present in the reaction chamber when the first detectably labeled nucleotide is being incorporated into the sequencing primer. In some embodiments, other molecules of the first detectably labeled nucleotide that have not incorporated into a template nucleotide/sequencing primer duplex immobilized at a particular location are not removed when the second detectably labeled nucleotide is incorporated into the extended sequencing primer. In fact, the second detectably labeled nucleotide can be a molecule of the first detectably labeled nucleotide that has not incorporated. For instance, the first detectably labeled nucleotide can be an A nucleotide, and another A nucleotide can be the second detectably labeled nucleotide. Thus, for a template nucleotide/sequencing primer duplex at a given location, it can be said that detectably labeled nucleotides are continuously incorporated in a noncyclic manner.

In any of the embodiments herein, the template nucleotide for a sequencing method disclosed herein can be in a decimated cluster (e.g., as shown in FIG. 2), where some template nucleotides in the same cluster have been deactivated such that the deactivated strands do not give rise to signals associated with nucleotide incorporation or nonincorporated events and the deactivated strands remain “dark” throughout the single nucleotide, real-time sequencing of strands within the cluster that are not deactivated.

### A. Nucleotides and Nucleotide Analogs

In some embodiments, a method disclosed herein comprises using one or more nucleotides or analogs thereof, including a native nucleotide or a nucleotide analog or modified nucleotide (e.g., labeled with one or more detectable labels). In some embodiments, a nucleotide analog comprises a nitrogenous base, five-carbon sugar, and phosphate group, wherein any component of the nucleotide may be modified and/or replaced. In some embodiments, a method disclosed herein may comprise but does not require using one or more non-incorporable nucleotides. Non-incorporable nucleotides may be modified to become incorporable at any point during the sequencing method.

Nucleotide analogs include, but are not limited to, alpha-phosphate modified nucleotides, alpha-beta nucleotide analogs, beta-phosphate modified nucleotides, beta-gamma nucleotide analogs, gamma-phosphate modified nucleotides, caged nucleotides, or ddNTPs. Examples of nucleotide analogs are described in U.S. Pat. No. 8,071,755, which is incorporated by reference herein in its entirety.

In some embodiments, a method disclosed herein may comprise but does not require using terminators that reversibly prevent nucleotide incorporation at the 3′-end of the primer. One type of reversible terminator is a 3′-O-blocked reversible terminator. Here the terminator moiety is linked to the oxygen atom of the 3′-OH end of the 5-carbon sugar of a nucleotide. For example, U.S. Pat. Nos. 7,544,794 and 8,034,923 (the disclosures of these patents are incorporated by reference) describe reversible terminator dNTPs having the 3′-OH group replaced by a 3′-ONH₂ group. Another type of reversible terminator is a 3′-unblocked reversible terminator, wherein the terminator moiety is linked to the nitrogenous base of a nucleotide. For example, U.S. Pat. No. 8,808,989 (the disclosure of which is incorporated by reference) discloses particular examples of base-modified reversible terminator nucleotides that may be used in connection with the methods described herein. Other reversible terminators that similarly can be used in connection with the methods described herein include those described in U.S. Pat. Nos. 7,956,171, 8,071,755, and 9,399,798, herein incorporated by reference.

In some embodiments, a method disclosed herein may comprise but does not require using nucleotide analogs having terminator moieties that irreversibly prevent nucleotide incorporation at the 3′-end of the primer. Irreversible nucleotide analogs include 2′, 3′-dideoxynucleotides, ddNTPs (ddGTP, ddATP, ddTTP, ddCTP). Dideoxynucleotides lack the 3′-OH group of dNTPs that is essential for polymerase-mediated synthesis.

In some embodiments, a method disclosed herein may comprise but does not require using non-incorporable nucleotides comprising a blocking moiety that inhibits or prevents the nucleotide from forming a covalent linkage to a second nucleotide (3′-OH of a primer) during the incorporation step of a nucleic acid polymerization reaction. The blocking moiety can be removed from the nucleotide, allowing for nucleotide incorporation.

In some embodiments, a method disclosed herein may comprise but does not require using 1, 2, 3, 4 or more nucleotide analogs present in the SBS reaction. In some embodiments, a nucleotide analog is replaced, diluted, or sequestered during an incorporation step. In some embodiments, a nucleotide analog is replaced with a native nucleotide. In some embodiments, a nucleotide analog is modified during an incorporation step. The modified nucleotide analog can be similar to or the same as a native nucleotide.

In some embodiments, a method disclosed herein may comprise but does not require using a nucleotide analog having a different binding affinity for a polymerase than a native nucleotide. In some embodiments, a nucleotide analog has a different interaction with a next base than a native nucleotide. Nucleotide analogs and/or non-incorporable nucleotides may base-pair with a complementary base of a template nucleic acid.

In some embodiments, one or more nucleotides can be labeled with distinguishing and/or detectable tags or labels. The tags may be distinguishable by means of their differences in fluorescence, Raman spectrum, charge, mass, refractive index, luminescence, length, or any other measurable property. The tag may be attached to one or more different positions on the nucleotide, so long as the fidelity of binding to the polymerase-nucleic acid complex is sufficiently maintained to enable identification of the complementary base on the template nucleic acid correctly. In some embodiments, the tag is attached to the nucleobase of the nucleotide. Alternatively, a tag is attached to the gamma phosphate position of the nucleotide.

Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes. The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. In some embodiments, the detectable label is bound to another moiety, for example, a nucleotide or nucleotide analog, and can include a fluorescent, a colorimetric, or a chemiluminescent label.

In some embodiments, a detectable label can be attached to another moiety, for example, a nucleotide or nucleotide analog. In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor®430, Alexa Fluor®488, Alexa Fluor®532, Alexa Fluor®546, Alexa Fluor®555, Alexa Fluor®568, Alexa Fluor®594, Alexa Fluor®633, Alexa Fluor®647, Alexa Fluor®660, Alexa Fluor®680, Alexa Fluor®700, Alexa Fluor®750, Allophycocyanin (APC), AMCA/AMCA-X, 7-Aminoactinomycin D (7-AAD), 7- Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, Cl-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DiA (4-Di-16-ASP), DiD (DilC18(5)), DIDS, Dil (DilC18(3)), DiO (DiOC18(3)), DiR (DilC18(7)), Di-4 ANEPPS, Di-8 ANEPPS, DM-NERF (4.5-6.5 pH), DsRed (Red Fluorescent Protein), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™ Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/PO-PRO™-1, pQpO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed®, SYTO® 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 542, ATTO 550, ATTO 565, ATTO Rho101, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. The label can emit a signal or alter a signal delivered to the label so that the presence or absence of the label can be detected. In some cases, coupling may be via a linker, which may be cleavable, such as photo-cleavable (e.g., cleavable under ultra-violet light), chemically-cleavable (e.g., via a reducing agent, such as dithiothreitol (DTT), tris(2-carboxyethyl)phosphine (TCEP)) or enzymatically cleavable (e.g., via an esterase, lipase, peptidase, or protease).

### B. Polymerases

Polymerases that may be used to carry out the disclosed techniques include naturally-occurring polymerases and any modified variations thereof, including, but not limited to, mutants, recombinants, fusions, genetic modifications, chemical modifications, synthetics, and analogs. Naturally occurring polymerases and modified variations thereof are not limited to polymerases that retain the ability to catalyze a polymerization reaction. In some embodiments, the naturally occurring and/or modified variations thereof retain the ability to catalyze a polymerization reaction. In some embodiments, the naturally-occurring and/or modified variations have special properties that enhance their ability to sequence DNA, including enhanced binding affinity to nucleic acids, reduced binding affinity to nucleic acids, enhanced catalysis rates, reduced catalysis rates, etc. Mutant polymerases include polymerases wherein one or more amino acids are replaced with other amino acids (naturally or non-naturally occurring), and insertions or deletions of one or more amino acids.

In some embodiments, a method disclosed herein may comprise but does not require using modified polymerases containing an external tag (e.g., an exogenous detectable label), which can be used to monitor the presence and interactions of the polymerase. In some embodiments, intrinsic signals from the polymerase can be used to monitor their presence and interactions. Thus, the provided methods can include monitoring the interaction of the polymerase, nucleotide and template nucleic acid through detection of an intrinsic signal from the polymerase. In some embodiments, the intrinsic signal is a light scattering signal. For example, intrinsic signals include native fluorescence of certain amino acids such as tryptophan.

In some embodiments, a method disclosed herein may comprise using an unlabeled polymerase, and monitoring is performed in the absence of an exogenous detectable label associated with the polymerase. Some modified polymerases or naturally occurring polymerases, under specific reaction conditions, may incorporate only single nucleotides and may remain bound to the primer-template after the incorporation of the single nucleotide.

In some embodiments, a method disclosed herein may comprise using an polymerase unlabeled with an exogenous detectable label (e.g., a fluorescent label). The label can be chemically linked to the structure of the polymerase by a covalent bond after the polymerase has been at least partially purified using protein isolation techniques. For example, the exogenous detectable label can be chemically linked to the polymerase using a free sulfhydryl or a free amine moiety of the polymerase. This can involve chemical linkage to the polymerase through the side chain of a cysteine residue, or through the free amino group of the N-terminus. In certain preferred embodiments, a fluorescent label attached to the polymerase is useful for locating the polymerase, as may be important for determining whether or not the polymerase has localized to a spot on an array corresponding to immobilized primed template nucleic acid. The fluorescent signal need not, and in some embodiments does not change absorption or emission characteristics as the result of binding any nucleotide. In some embodiments, the signal emitted by the labeled polymerase is maintained uniformly in the presence and absence of any nucleotide being investigated as a possible next correct nucleotide.

The term polymerase and its variants, as used herein, also refers to fusion proteins comprising at least two portions linked to each other, for example, where one portion comprises a peptide that can catalyze the polymerization of nucleotides into a nucleic acid strand is linked to another portion that comprises a second moiety, such as, a reporter enzyme or a processivity-modifying domain. For example, T7 DNA polymerase comprises a nucleic acid polymerizing domain and a thioredoxin binding domain, wherein thioredoxin binding enhances the processivity of the polymerase. Absent the thioredoxin binding, T7 DNA polymerase is a distributive polymerase with processivity of only one to a few bases. Although DNA polymerases differ in detail, they have a similar overall shape of a hand with specific regions referred to as the fingers, the palm, and the thumb; and a similar overall structural transition, comprising the movement of the thumb and/or finger domains, during the synthesis of nucleic acids.

DNA polymerases include, but are not limited to, bacterial DNA polymerases, eukaryotic DNA polymerases, archaeal DNA polymerases, viral DNA polymerases and phage DNA polymerases. Bacterial DNA polymerases include E. coli DNA polymerases I, II and III, IV and V, the Klenow fragment of E. coli DNA polymerase, Clostridium stercorarium (Cst) DNA polymerase, Clostridium thermocellum (Cth) DNA polymerase and Sulfolobus solfataricus (Sso) DNA polymerase. Eukaryotic DNA polymerases include DNA polymerases α, β, γ, δ, ϵ, η, ζ, λ, σ, μ, and κ, as well as the Rev1 polymerase (terminal deoxycytidyl transferase) and terminal deoxynucleotidyl transferase (TdT). Viral DNA polymerases include T4 DNA polymerase, phi-29 DNA polymerase, GA-1, phi-29-like DNA polymerases, PZA DNA polymerase, phi-15 DNA polymerase, Cpl DNA polymerase, Cp7 DNA polymerase, T7 DNA polymerase, and T4 polymerase. Other DNA polymerases include thermostable and/or thermophilic DNA polymerases such as DNA polymerases isolated from Thermus aquaticus (Taq) DNA polymerase, Thermus filiformis (Tfi) DNA polymerase, Thermococcus zilligi (Tzi) DNA polymerase, Thermus thermophilus (Tth) DNA polymerase, Thermus flavusu (Tfl) DNA polymerase, Pyrococcus woesei (Pwo) DNA polymerase, Pyrococcus furiosus (Pfu) DNA polymerase and Turbo Pfu DNA polymerase, Thermococcus litoralis (Tli) DNA polymerase, Pyrococcus sp. GB-D polymerase, Thermotoga maritima (Tma) DNA polymerase, Bacillus stearothermophilus (Bst) DNA polymerase, Pyrococcus Kodakaraensis (KOD) DNA polymerase, Pfx DNA polymerase, Thermococcus sp. JDF-3 (JDF-3) DNA polymerase, Thermococcus gorgonarius (Tgo) DNA polymerase, Thermococcus acidophilium DNA polymerase; Sulfolobus acidocaldarius DNA polymerase; Thermococcus sp. go N-7 DNA polymerase; Pyrodictium occultum DNA polymerase; Methanococcus voltae DNA polymerase; Methanococcus thermoautotrophicum DNA polymerase; Methanococcus jannaschii DNA polymerase; Desulfurococcus strain TOK DNA polymerase (D. Tok Pol); Pyrococcus abyssi DNA polymerase; Pyrococcus horikoshii DNA polymerase; Pyrococcus islandicum DNA polymerase; Thermococcus fumicolans DNA polymerase; Aeropyrum pernix DNA polymerase; and the heterodimeric DNA polymerase DP1/DP2. Engineered and modified polymerases also are useful in connection with the disclosed techniques. For example, modified versions of the extremely thermophilic marine archaea Thermococcus species 9° N (e.g., Therminator DNA polymerase from New England BioLabs Inc.; Ipswich, Mass.) can be used. Still other useful DNA polymerases, including the 3PDX polymerase are disclosed in U.S. Pat. No. 8,703,461, the disclosure of which is incorporated by reference in its entirety.

RNA polymerases include, but are not limited to, viral RNA polymerases such as T7 RNA polymerase, T3 polymerase, SP6 polymerase, and K11 polymerase; Eukaryotic RNA polymerases such as RNA polymerase I, RNA polymerase II, RNA polymerase III, RNA polymerase IV, and RNA polymerase V; and Archaea RNA polymerase.

Reverse transcriptases include, but are not limited to, HIV-1 reverse transcriptase from human immunodeficiency virus type 1 (PDB 1HMV), HIV-2 reverse transcriptase from human immunodeficiency virus type 2, M-MLV reverse transcriptase from the Moloney murine leukemia virus, AMV reverse transcriptase from the avian myeloblastosis virus, and Telomerase reverse transcriptase that maintains the telomeres of eukaryotic chromosomes.

### C. Sequencing Reactions

In some embodiments of a sequencing-by-synthesis (SBS) method disclosed herein, a first labeled nucleotide that has been incorporated is not deactivated (e.g., by removal and/or photobleaching of the label) prior to the introduction and/or incorporation of the next, second labeled nucleotide. The first and second labeled nucleotides can comprise the same base or different bases. The first and second labeled nucleotides can be introduced into a sequencing reaction mix simultaneously or at different time points in any order. Further, the first and second labeled nucleotides can be introduced by itself (e.g., in a suitable solvent such as water) or in a mixture with another sequencing reagent, such as one or more other labeled nucleotides and/or one or more unlabeled nucleotides. The first and second labeled nucleotides can also comprise the same base or different bases. In some embodiments, nucleotides that have not been incorporated at a residue corresponding to a base in the template nucleic acid (e.g., because the first labeled nucleotide has been incorporated at that residue) are not removed from the sequencing reaction mix prior to the introduction and/or incorporation of the second labeled nucleotide. In some embodiments, the first and second labeled nucleotides (and optionally labeled nucleotides for interrogating subsequent bases in the template) are provided in the same sequencing reaction mix, and the first, second, and optionally any subsequent labeled nucleotide(s) are incorporated sequentially in a continuous manner.

Thus, unlike existing SBS methods, some embodiments of the method disclosed herein use continuous introduction and/or incorporation of nucleotides (e.g., fluorescently labeled A, T, C, and/or G nucleotides) without the need of label deactivation and/or wash steps in between sequential incorporation events for a given template nucleic acid molecule to be sequenced. Rather, in some embodiments, label deactivation (e.g., by cleaving and/or photobleaching the label) of a first incorporated nucleotide may occur stochastically throughout the continuous nucleotide incorporation process, for instance, prior to, during, or after the incorporation of a second, third, fourth, or a subsequent labeled nucleotide.

Nucleic acid sequencing reaction mixtures, or simply “reaction mixtures,” typically include reagents that are commonly present in polymerase based nucleic acid synthesis reactions. The reaction mixture can include other molecules including, but not limited to, enzymes. In some embodiments, the reaction mixture comprises any reagents or biomolecules generally present in a nucleic acid polymerization reaction. Reaction components may include, but are not limited to, salts, buffers, small molecules, detergents, crowding agents, metals, and ions. In some embodiments, properties of the reaction mixture may be manipulated, for example, electrically, magnetically, and/or with vibration.

The provided methods herein may further comprise but do not require one or more wash steps; a temperature change; a mechanical vibration; a pH change; or an optical stimulation that is not dye illumination or photobleaching. In some embodiments, the wash step comprises contacting the substrate and the nucleic acid molecule, the primer, and/or the polymerase with one of more buffers, detergents, protein denaturants, proteases, oxidizing agents, reducing agents, or other agents capable of crosslinking or releasing crosslinks, e.g., crosslinks within a polymerase or crosslinks between a polymerase and nucleic acid. Methods and compositions for nucleic acid sequencing are known, for example, as described in U.S. Pat. Nos. 10,246,744 and 10,844,428, incorporated herein by reference in their entireties for all purposes.

Reaction mixture reagents can include, but are not limited to, enzymes (e.g., polymerase), dNTPs, template nucleic acids, primer nucleic acids, salts, buffers, small molecules, co-factors, metals, and ions. The ions may be catalytic ions, divalent catalytic ions, non-catalytic ions, non-covalent metal ions, or a combination thereof. The reaction mixture can include salts, such as NaCl, KCl, potassium acetate, ammonium acetate, potassium glutamate, or NH₄Cl or the like, that ionize in aqueous solution to yield monovalent cations. The reaction mixture can include a source of ions, such as Mg²⁺, Mn²⁺, Co²⁺, Cd²⁺, and/or Ba²⁺ ions. The reaction mixture can include tin, Ca²⁺, Zn²⁺, Cu²⁺, Co²⁺, Fe²⁺, and/or Ni²⁺, or other divalent non-catalytic metal cations. In some embodiments, the reaction mixture can include metal cations that may inhibit formation of phosphodiester bonds between the primed template nucleic acid molecule and the cognate nucleotide. In some embodiments, the metal cations can be used (e.g., at a suitable concentration) to slow down but not completely inhibit or prevent nucleotide incorporation, thereby reducing multiple nucleotide incorporation events in a single detection window.

In some embodiments, the sequencing reaction conditions comprise contacting the nucleic acid molecule and the primer with a buffer that regulates osmotic pressure. In some embodiments, the reaction mixture comprises a buffer that regulates osmotic pressure. In some embodiments, the buffer is a high salt buffer that includes a monovalent ion, such as a monovalent metal ion (e.g., potassium ion or sodium ion) at a concentration of from about 50 to about 1,500 mM. Salt concentrations in the range of from about 100 to about 1,500 mM, or from about 200 to 1,000 mM may also be used. In some embodiments, the buffer further comprises a source of glutamate ions (e.g., potassium glutamate). In some embodiments, the buffer comprises a stabilizing agent. In some embodiments, the stabilizing agent is a non-catalytic metal ion (e.g., a divalent non-catalytic metal ion). Non-catalytic metal ions useful in this context include, but are not limited to, calcium, strontium, scandium, titanium, vanadium, chromium, iron, cobalt, nickel, copper, zinc, gallium, germanium, arsenic, selenium, rhodium, europium, and/or terbium. In some embodiments, the non-catalytic metal ion is strontium, tin, or nickel. In some embodiments, the sequencing reaction mixture comprises strontium chloride or nickel chloride. In some embodiments, the stabilizing agent can be used (e.g., at a suitable concentration) to slow down but not completely inhibit or prevent nucleotide incorporation, thereby reducing multiple nucleotide incorporation events in a single detection window.

The buffer can include Tris, Tricine, HEPES, MOPS, ACES, MES, phosphate-based buffers, and acetate-based buffers. The reaction mixture can include chelating agents such as EDTA, EGTA, and the like. In some embodiments, the reaction mixture includes cross-linking reagents.

In some embodiments, the interaction between the polymerase and template nucleic acid may be manipulated by modulating sequencing reaction parameters such as ionic strength, pH, temperature, or any combination thereof, or by the addition of a destabilizing agent to the reaction. In some embodiments, the destabilizing agent can be used (e.g., at a suitable concentration) to slow down but not completely inhibit or prevent nucleotide incorporation, thereby reducing multiple nucleotide incorporation events in a single detection window.

In some embodiments, high salt (e.g., 50 to 1,500 mM) and/or pH changes are utilized to destabilize a complex between the polymerase and template nucleic acid. In some embodiments, the reaction conditions favor the stabilization of a complex among the polymerase, the template nucleic acid, and a labeled nucleotide. By way of example, the pH of the reaction mixture can be adjusted from 4.0 to 10.0 to favor the stabilization of a complex among the polymerase, the template nucleic acid, and a labeled nucleotide. In some embodiments, the pH of the reaction mixture is from 4.0 to 6.0. In some embodiments, the pH of the reaction mixture is 6.0 to 10.0. In some embodiments, a suitable salt concentration and/or a suitable pH can be selected to slow down but not completely inhibit or prevent nucleotide incorporation, thereby reducing multiple nucleotide incorporation events in a single detection window.

In some embodiments, the reaction mixture comprises a competitive inhibitor, where the competitive inhibitor may reduce the occurrence of multiple incorporations events in a detection window. In one embodiment, the competitive inhibitor is a non-incorporable nucleotide. In an embodiment, the competitive inhibitor is an aminoglycoside. The competitive inhibitor is capable of replacing either the nucleotide or the catalytic metal ion in the active site, such that the competitive inhibitor occupies the active site preventing or slowing down a nucleotide incorporation. In some embodiments, both an incorporable nucleotide and a competitive inhibitor are introduced, such that the ratio of the incorporable nucleotide and the inhibitor can be adjusted to modulate the rate of incorporation of a single nucleotide at the 3′-end of the primer. In some embodiments, the competitive inhibitor can be used (e.g., at a low concentration) to slow down but not completely inhibit or prevent nucleotide incorporation, thereby reducing multiple nucleotide incorporation events in a single detection window.

In some embodiments, the reaction mixture comprises at least one nucleotide molecule that is a non-incorporable nucleotide. In some embodiments, the reaction mixture comprises one or more nucleotide molecules incapable of incorporation into the primer of the primed template nucleic acid molecule. Such nucleotides incapable of incorporation include, for example, monophosphate nucleotides. For example, the nucleotide may contain modifications to the triphosphate group that make the nucleotide non-incorporable. Examples of non-incorporable nucleotides may be found in U.S. Pat. No. 7,482,120, which is incorporated by reference herein in its entirety. In some embodiments, the primer may not contain a free hydroxyl group at its 3′-end, thereby rendering the primer incapable of incorporating any nucleotide, and, thus, making any nucleotide non-incorporable. In some embodiments, the primer may be processed such that it contains a free hydroxyl group at its 3′-end to allow nucleotide incorporation. In some embodiments, the non-incorporable nucleotide can be used (e.g., at a low concentration) to slow down but not completely inhibit or prevent nucleotide incorporation, thereby reducing multiple nucleotide incorporation events in a single detection window.

In some embodiments, the reaction mixture comprises at least one nucleotide molecule that is incorporable but is incorporated at a slower rate compared to a corresponding naturally-occurring nucleoside triphosphate (e.g., NTP or dNTP). Such nucleotides incorporable at a slower rate may include, for example, diphosphate nucleotides. For example, the nucleotide may contain modifications to the triphosphate group that make the nucleotide incorporable at a slower rate. In some embodiments, the nucleotide incorporable at a slower rate can be used to slow down but not completely inhibit or prevent nucleotide incorporation, thereby reducing multiple nucleotide incorporation events in a single detection window.

In some embodiments, the reaction mixture comprises a polymerase inhibitor. In some embodiments, the polymerase inhibitor is a pyrophosphate analog. In some embodiments, the polymerase inhibitor is an allosteric inhibitor. In some embodiments, the polymerase inhibitor is a DNA or an RNA aptamer. In some embodiments, the polymerase inhibitor competes with a catalytic-ion binding site in the polymerase. In some embodiments, the polymerase inhibitor is a reverse transcriptase inhibitor. The polymerase inhibitor may be an HIV-1 reverse transcriptase inhibitor or an HIV-2 reverse transcriptase inhibitor. The HIV-1 reverse transcriptase inhibitor may be a (4/6-halogen/MeO/EtO-substituted benzo[d]thiazol-2-yl)thiazolidin-4-one. In some embodiments, the polymerase inhibitor can be used (e.g., at a low concentration) to slow down but not completely inhibit or prevent nucleotide incorporation, thereby reducing multiple nucleotide incorporation events in a single detection window.

In some embodiments, the contacting step is facilitated by the use of a chamber such as a flow cell. The methods and apparatus described herein may employ next generation sequencing technology (NGS), which allows massively parallel sequencing. In some embodiments, single DNA molecules are sequenced in a massively parallel fashion within a reaction chamber. A flow cell may be used but is not necessary. Flowing liquid reagents through the flow cell, which contains an interior solid support surface (e.g., a planar surface), conveniently permits reagent exchange. Immobilized to the interior surface of the flow cell is one or more primed template nucleic acids to be sequenced or interrogated using the procedures described herein. Typical flow cells will include microfluidic valving that permits delivery of liquid reagents (e.g., components of the “reaction mixtures” discussed herein) to an entry port. Liquid reagents can be removed from the flow cell by exiting through an exit port.

In some embodiments, a reaction chamber disclosed herein can comprise a reagent wall, an imaging area, and optionally an outlet configured to remove molecules of one or more of the polymerase, the first detectably labeled nucleotide, the second detectably labeled nucleotide, and/or one or more other reagents from the imaging area. In some embodiments, the device may comprise one or more vents but no outlet or exit port for the reaction mixture. In some embodiments, a method disclosed herein does not comprise a step of removing liquid reagents through an outlet or exit port, e.g., from a reaction chamber such as a flow cell.

The methods disclosed herein may but do not need to be used in combination with any NGS sequencing methods. The sequencing technologies of NGS include but are not limited to pyrosequencing, sequencing-by-synthesis with reversible dye terminators, sequencing by oligonucleotide probe ligation, and ion semiconductor sequencing. Nucleic acids such as DNA or RNA from individual samples can be sequenced individually (singleplex sequencing) or nucleic acids such as DNA or RNA from multiple samples can be pooled and sequenced as indexed genomic molecules (multiplex sequencing) on a single sequencing run, to generate up to several hundred million reads of sequences. Examples of sequencing technologies that can be used to obtain the sequence information according to the present method are further described here.

Some sequencing technologies are available commercially, such as the sequencing-by-hybridization platform from Affymetrix Inc. (Sunnyvale, Calif.) and the sequencing-by-synthesis platforms from 454 Life Sciences (Bradford, Conn.), Illumina/Solexa (Hayward, Calif.) and Helicos Biosciences (Cambridge, Mass.), and the sequencing-by-ligation platform from Applied Biosystems (Foster City, Calif.). In addition to the single molecule sequencing performed using sequencing-by-synthesis of Helicos Biosciences, other single molecule sequencing technologies include, but are not limited to, the SMRT™ technology of Pacific Biosciences, the ION TORRENT™ technology, and nanopore sequencing developed for example, by Oxford Nanopore Technologies.

While the automated Sanger method is considered as a ‘first generation’ technology, Sanger sequencing including the automated Sanger sequencing, can also be employed in the methods described herein. Additional suitable sequencing methods include, but are not limited to nucleic acid imaging technologies, e.g., atomic force microscopy (AFM) or transmission electron microscopy (TEM).

In some embodiments, the disclosed methods may be used in combination with massively parallel sequencing of nucleic acid molecules using Illumina's sequencing-by-synthesis and reversible terminator-based sequencing chemistry. In some implementation, a method disclosed herein can use a flow cell having a glass slide with lanes.

After sequencing of nucleic acid molecules, sequence reads of predetermined length, e.g., at least about 15 bp, are localized by mapping (alignment) to a known reference sequence or genome (e.g., viral sequences or genomes). A number of computer algorithms are available for aligning sequences, including without limitation BLAST, BLITZ, FASTA, BOWTIE, or ELAND (Illumina, Inc., San Diego, Calif., USA).

In one illustrative, but non-limiting, embodiment, the methods described herein may comprise obtaining sequence information for the nucleic acids in a test sample, using single molecule sequencing technology similar to the Helicos True Single Molecule Sequencing (tSMS) technology. In the tSMS technique, a DNA sample is cleaved into strands of approximately 100 to 200 nucleotides, and a polyA sequence is added to the 3′ end of each DNA strand. Each strand is labeled by the addition of a fluorescently labeled adenosine nucleotide. The DNA strands are then hybridized to a flow cell, which contains millions of oligo-T capture sites that are immobilized to the flow cell surface. In some embodiments the templates can be at a density of about 100 million templates/cm². The flow cell is then loaded into an instrument, e.g., HeliScope™ sequencer, and a laser illuminates the surface of the flow cell, revealing the position of each template. A CCD camera can map the position of the templates on the flow cell surface. The template fluorescent label is then cleaved and washed away. The sequencing reaction begins by introducing a DNA polymerase and a fluorescently labeled nucleotide. The oligo-T nucleic acid serves as a primer. The polymerase incorporates the labeled nucleotides to the primer in a template directed manner. The polymerase and unincorporated nucleotides are removed. The templates that have directed incorporation of the fluorescently labeled nucleotide are discerned by imaging the flow cell surface. After imaging, a cleavage step removes the fluorescent label, and the process is repeated with other fluorescently labeled nucleotides until the desired read length is achieved. Sequence information is collected with each nucleotide addition step. Whole genome sequencing by single molecule sequencing technologies excludes or typically obviates PCR-based amplification in the preparation of the sequencing libraries, and the methods allow for direct measurement of the sample, rather than measurement of copies of that sample.

In another illustrative, but non-limiting, embodiment, the methods described herein may comprise obtaining sequence information for the nucleic acids in the test sample, similar to the single molecule, real-time (SMRT™) sequencing technology of Pacific Biosciences. In SMRT sequencing, the continuous incorporation of dye-labeled nucleotides is imaged during DNA synthesis. Single DNA polymerase molecules are attached to the bottom surface of individual zero-mode wavelength detectors (ZMW detectors) that obtain sequence information while phospholinked nucleotides are being incorporated into the growing primer strand. A ZMW detector includes a confinement structure that enables observation of incorporation of a single nucleotide by DNA polymerase against a background of fluorescent nucleotides that rapidly diffuse in an out of the ZMW (e.g., in microseconds). It typically takes several milliseconds to incorporate a nucleotide into a growing strand. During this time, the fluorescent label is excited and produces a fluorescent signal, and the fluorescent tag is cleaved off. Measurement of the corresponding fluorescence of the dye indicates which base was incorporated. The process is repeated to provide a sequence.

In some embodiments, the provided sequencing methods disclosed herein may regulate polymerase interaction with the nucleotides and template nucleic acid (as well as rate of nucleotide incorporation) in a manner that reveals the identity of the next base while controlling the chemical addition of a nucleotide. In some embodiments, the SBS reaction condition comprises a plurality of primed template nucleic acids, polymerases, nucleotides, or any combination thereof. In some embodiments, the plurality of nucleotides comprises 1, 2, 3, 4, or more types of different nucleotides, for example dATP, dTTP (or dUTP), dGTP, and dCTP. In some embodiments, the plurality of template nucleic acids are single molecules immobilized on a substrate for single molecule sequencing.

In some embodiments, the method can further comprise contacting the nucleic acid molecule with the substrate to immobilize the nucleic acid molecule. In some embodiments, the nucleic acid molecule can be immobilized at a density of one molecule per at least about 250 nm², at least about 200 nm², at least about 150 nm², at least about 100 nm², at least about 90 nm², at least about 80 nm², at least about 70 nm², at least about 60 nm², at least about 50 nm², at least about 40 nm², at least about 30 nm², at least about 20 nm², at least about 10 nm², at least about 5 nm², or in between any two of the aforementioned values. Methods and compositions for arraying biomolecules on a substrate, e.g., as described in US 2005/0042649 (incorporated herein by reference in its entirety for all purposes), may be used in methods disclosed herein.

In some embodiments, nucleic acid molecules, polymerase molecules, and/or sequencing primers can be provided on the substrate for super-resolution signal detection. For instance, two nucleic acid molecules to be sequenced may be at two spots near each other. If only one spot is emitting at any one time, a localization based technique may be used to resolve the spot locations to sub-diffraction limited resolution, thereby assigning detected signals (e.g., emissions) to different molecules/strands under synthesis. In such cases, nucleic acid molecules to be sequenced may be packed on the substrate at a density of about one molecule per 20 nm², one molecule per 15 nm², one molecule per 10 nm², at least about 5 nm², or even higher density.

In some embodiments, the detectable labels may comprise one or more labels that blink which may be used to achieve super-resolution localization of nucleic acid strands being sequenced during sequencing at the single molecule level. In some embodiments, labels with differing blinking characteristics may be used for labeling one or more nucleotides. In some embodiments, the detectable labels may comprise one or more labels that exhibit stochastic blinking (also known as photoluminescence intermittence), such as quantum dots. The phenomenon of blinking may be due to high excitation power resulting in a local electric field, nonradiative Auger recombination, and/or surface trap induced recombination. Blinking may be photo-induced or spontaneous, for instance, as described in Stefani et al., “Quantification of photoinduced and spontaneous quantum-dot luminescence blinking,” Physical Review B 72, 125304 (2005), incorporated herein by reference in its entirety for all purposes. Inherent quantum dot blinking is generally believed to interfere with fluorescence quenching assays and techniques are available to limit intermittent fluorescence. In some embodiments herein, labels (such as quantum dots) that blink may be used, for instance, in cases where nucleic acid molecule density on the substrate is high. In examples where two nucleotides with blinking labels or one nucleotide with a blinking label and another with a non-blinking label are incorporated at two nearby spots, signals detected at one or more time points where only one of the two labels is emitting may be used to resolve the two nearby spot locations.

In some embodiments, a subset of nucleic acid molecules (e.g., nucleic acid strands to be sequenced) on the substrate may be active at one or more time points. In some embodiments, at any one time, a first subset of nucleic acid molecules on the substrate is active (e.g., allowing nucleotide incorporation into a sequencing primer using a single-stranded sequence as template) while a second subset of nucleic acid molecules on the substrate is inactive (e.g., not allowing nucleotide incorporation into a sequencing primer using a single-stranded sequence as template). In some embodiments, at one or more time points, a first subset of nucleic acid molecules on the substrate is activated (e.g., by a first set of polymerase and/or primer molecules) for nucleotide incorporation, while a second subset of nucleic acid molecules on the substrate is not activated (e.g., by the first set of polymerase and/or primer molecules), thus only signals associated with the first subset of nucleic acid molecules are detected. At one or more other time points, the second subset of nucleic acid molecules on the substrate is activated (e.g., by a second set of polymerase and/or primer molecules) for nucleotide incorporation, while the first subset of nucleic acid molecules on the substrate is not activated (e.g., by the second set of polymerase and/or primer molecules), thus only signals associated with the second subset of nucleic acid molecules are detected. In some embodiments, the first and second sets of polymerase and/or primer molecules can be introduced at different time points, e.g., in sequential cycles with optional washing steps between cycles (e.g., to remove a set of polymerase and/or primer molecules for SBS of a first subset of strands before introducing the next set of polymerase and/or primer molecules for SBS of a second subset of strands). In some embodiments, regardless of whether a particular strand being sequenced is in the first subset or the second subset, nucleotide incorporation using the particular strand as template can occur in a non-cyclical manner as described herein.

In some embodiments, the substrate can comprise a bead, a planar substrate, a solid surface, a flow cell, a semiconductor chip, a well, a pillar, a chamber, a channel, a through hole, a nanopore, or any combination thereof. In some embodiments, the substrate can comprise a microwell, a micropillar, a microchamber, a microchannel, or any combination thereof.

### D. Signal Deactivation

In some embodiments, one or more of the incorporated nucleotides may be stochastically deactivated (e.g., by photobleaching and/or cleaving the labels) in a non-cyclically manner. In some embodiments, for a given labeled nucleotide, once the label is cleaved or deactivated, the signal intensity (if any remains) associated with the nucleotide no longer changes, e.g., in response to light that bleaches labels on other nucleotides. For instance, in one embodiment, after the fluorescent dye of a particular dye-labeled nucleotide is photobleached (thus fluorescence intensity associated with dye-labeled nucleotide decreases from a first intensity to a second, lower intensity), the photobleached dye-labeled nucleotide does not recover to the first fluorescence intensity. In some embodiments, the fluorescence intensity of the photobleached dye-labeled nucleotide remains at the second intensity which can be zero; in other words, the photobleached dye can go “dark,” e.g., its signal is below a certain threshold or undetectable and does not recover. In some embodiments, an increase in signal intensity due to a nucleotide incorporation event in a method disclosed herein is not detected as an increase due to a photobleached dye recovering from a bleached state. In some embodiments, a photobleached dye herein is prevented from recovering from a bleached state such that an increase in signal intensity is attributable to nucleotide incorporation rather than recovery from photobleaching. In some embodiments, for each label that has been deactivated (e.g., photobleached), the deactivation is complete in that the deactivated label does not recover. In some embodiments, labels at multiple locations (some of which may comprise the same label and others may comprise different labels) are not deactivated (e.g., photobleached) at the same time or in the same time window (e.g., in the same cycle). Rather, in a method disclosed herein, labels at different locations may be deactivated stochastically such that at a given time point or in a given time window, the labels at all locations of the substrate are not completely deactivated whereas for each label the signal deactivation is or will be complete (e.g., no signal recovery from a deactivated state).

In some embodiments where recovery from a deactivated state (e.g., after photobleaching) may occur, a recovery probability may be modeled and used during base calling. In some embodiments, the recovery probability is modeled using a reference based correction. Dye recovery from photobleaching has been described, for instance, by Braslavsky et al., “Sequence information can be obtained from single DNA molecules,” PNAS 100(7): 3960-64 (2003), incorporated herein by reference in its entirety for all purposes.

In some embodiments, stepwise changes over time in fluorophore emission (e.g., stepwise increases and/or decreases in signal intensity) at the particular spots can be detected and/or monitored. An increase in signal intensity (e.g., due to a nucleotide incorporation) and/or a decrease in signal (e.g., due to a photobleaching event) at a particular spot and in a given time window or time point (e.g., an imaging window in terms of frame/exposure) may partially or completely offset one another. In some embodiments, incorporation of a labeled nucleotide results in an increase in signal intensity characteristic of the label and/or the base of the incorporated labeled nucleotide. For instance, a nucleotide can be labeled with a label having a signal intensity characteristic of the base in that nucleotide, which can be distinguished from the signal intensity of the label on another nucleotide having a different base. In some embodiments, signal deactivation (e.g., by cleaving and/or photobleaching the label) of a labeled nucleotide results in a decrease in signal intensity characteristic of the label and/or the base of the signal-deactivated labeled nucleotide.

### E. Deconvolution and Basecalling

In some embodiments, each type of nucleotide (e.g., nucleotides comprising A, T/U, C, or G) can be labelled with a different fluorophore such that emissions of a particular fluorophore would be passed by one filter and rejected by all others. An exemplary high-throughput sequencing platform for real-time monitoring of biological processes by multicolor single-molecule fluorescence is described in Chen et al., PNAS 111 (2) 664-669 (2014) which is incorporated herein by reference in its entirety for all purposes.

In some embodiments, provided herein is a method comprising the use of labels with differing intensities (e.g., brightness) over a range of wavelengths. When combined with an appropriate filter, different dyes can be registered as different intensities using a single fixed filter and camera. This is advantageous as it results in a simpler and cheaper optical system. Such a labeling scheme may be used in a real-time context (e.g., cycle-less, no terminators) where each nucleotide incorporates and bleaches stochastically. For instance, dyes on incorporated nucleotides may not be completely bleached (or otherwise stochastically removed) before a subsequent nucleotide is incorporated. In some aspects, composition of bases (e.g., contiguous nucleic acid sequences) can be determined in a real-time sequencing approach, where nucleotides incorporate stochastically and labels bleach stochastically. In some embodiments, imaging is continuous in order to observe all incorporation events. In some cases, the average incorporation rate is tuned (e.g., through nucleotide concentration and/or polymerase activity) such that it is unlikely that multiple incorporations occur in a single frame. Similarly, the photobleaching rate can also be tuned (e.g., though laser intensity or oxygen scavenging additives).

While it's possible that photobleaching may occur in any order, incorporation and photobleaching events are matched. Photobleaching can occur with a fixed probability in each time point on the single molecule level. By tracking the incorporation events and photobleaching events, nucleic acid sequences of the strand being synthesized and the complementary template strand can be determined. A Hidden Markov Model (HMM) can be used to deconvolute the detected signal intensities over time in order to detect incorporation and bleaching events.

In some embodiments, the net change in signal intensity at the particular spot and the given time window or time point can be associated with the event(s) at the particular spot, for instance, incorporation of a new labeled nucleotide and photobleaching of one or more already incorporated labeled nucleotides. The one or more already incorporated labeled nucleotides may be at any distance from the newly incorporated labeled nucleotide, e.g., 0, 1, 2, 3, 4, 5, or more nucleotide residues apart. In some embodiments, the net change in signal intensity may be deconvoluted to one or more increases and/or one or more decreases in signal intensity that are characteristic of a nucleotide incorporation event (e.g., incorporation of a nucleotide labeled with a particular fluorophore) and a signal deactivation event (e.g., photobleaching of the same or another particular fluorophore), respectively.

In some embodiments, the deactivating step and/or the detecting step can be carried out as detectably labeled nucleotides are continuously provided to contact the nucleic acid molecule and/or the primer. In some embodiments, the detecting step is performed in real time as the nucleotide incorporation and signal deactivation (e.g., photobleaching) events occur. In some embodiments, the detecting step is not carried out using multiple switchable optical filters each for detecting a different detectable label. In some embodiments, the detecting step can be carried out using a dichroic filter to split optical signals into channels for detecting a different detectable label in each channel. In some embodiments, the detecting step can be carried out using total internal reflection fluorescence (TIRF) microscopy. In some embodiments, the signals in the detecting step can be compensated for background signal.

In some embodiments, nucleotide identification using the time trace can comprise probabilistically identifying the first, second, third, and/or fourth detectably labeled nucleotides. In some embodiments, the probabilistically identifying step can comprise assigning a state of signal intensity to each detectable label and decoding the time trace. In some embodiments, the state of signal intensity corresponds to a fixed value of signal intensity (e.g., sum of relative fluorescence over a range of excitation wavelengths). In some embodiments, the state of signal intensity corresponds to a range of signal intensities. In some embodiments, the state of signal intensity corresponds to a Gaussian distribution of signal intensities. In some embodiments, decoding the time trace may comprise pairing an incorporation event with a deactivation event of the detectable label of the nucleotide incorporated in the incorporation event. In some embodiments, decoding the time trace may comprise using a transition probability between two states of signal intensity, and the transition may comprise an incorporation event, a deactivation event (e.g., photobleaching), or an incorporation event and a deactivation event of the same label or different labels at a substrate location. In some embodiments, the transition probability between two states of signal intensity is fixed. In some embodiments, the transition probability between two states of signal intensity is fitted.

In some embodiments, a Hidden Markov Model (HMM) can be used to analyze the incorporation event(s) and/or the deactivation event(s) at one or more substrate locations by observing states of signal intensity and transitions between the states. In some embodiments, using the HMM comprises providing transition probabilities between states of signal intensity due to nucleotide incorporations and label bleaching where individual label bleaching is not expected to recover. For instance, the HMM can model a first state with two currently unbleached labels emitting, one on the incorporated first detectably labeled nucleotide and the other on the incorporated second detectably labeled nucleotide. In this example, the first state may transition into a second state where the label on the incorporated first detectably labeled nucleotide is bleached, or into a third state where the label on the incorporated second detectably labeled nucleotide is bleached. The first state may also transition into a fourth state due to incorporation of a third detectably labeled nucleotide, while the labels on the incorporated first and second detectably labeled nucleotides are not bleached. In some embodiments, decoding the time trace may comprise using the Viterbi algorithm for the HMM that represents incorporation and deactivation events.

In some embodiments, one or more of the sequence reads are about 10 bp, about 15 bp, about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. Mapping of the sequence reads can be achieved by comparing the sequence of the reads with the sequence of the reference to determine the origin of the sequenced nucleic acid molecule (e.g., from a virus such as a coronavirus, e.g., SARS-CoV-2). In some embodiments, the sequence reads can be mapped to one or more reference sequences or genomes. For instance, sequence reads generated using a method disclosed here for sequencing-based SARS-CoV-2 detection in a sample may map preferentially to a SARS-CoV-2 reference sequence or genome over a background of human sequences and other viral sequences. In some embodiments, certain degrees of mismatch (e.g., 0-2 mismatches per read, 2-5 mismatches per read, or 5 or more mismatches per read) may be allowed, and permitted degree of mismatch may be selected and/or adjusted depending on the application. In some embodiments, the degree of mismatch may be used to account for minor polymorphisms that may exist between the reference sequence or genome and the nucleic acid sequences in a mixed sample. In some embodiments, the degree of mismatch may be used to account for sequencing errors, e.g., technical errors rather than real differences in the sequence (e.g., sequence differences from two copies of a similar sequence in a sample). For instance, errors may be introduced in the manipulation of nucleic acids prior to or during single molecule sequencing reactions and/or may be introduced due to the intrinsic error rate of the polymerase used in the reactions.

In some embodiments, one or more of the sequence reads are no more than 100, no more than 90, no more than 80, no more than 70, no more than 60, no more than 50, no more than 40, no more than 30, no more than 20, no more than 15, or no more than 10 nucleotides in length. In some embodiments, the determined sequence of the nucleic acid molecule may be about 8, about 12, about 16, about 20, about 24, about 28, about 32, about 36, or about 40 nucleotides in length. In some embodiments, the determined sequence of the nucleic acid molecule may be between about 5 and about 50 nucleotides in length, such as between about 10 and about 35 nucleotides in length, or between about 15 and about 30 nucleotides in length.

In some embodiments, the methods described herein further comprise reporting information determined using the analytical methods and/or generating a report containing the information determined suing the analytical methods. For example, in some embodiments, the method further comprises reporting or generating a report containing related to the identification of a variant in a polynucleotide derived from a subject (e.g., from a virus that has infected the subject or within a subject's genome). Reported information or information within the report may be associated with sequencing reads mapped to a reference sequence, a detected variant (such as a detected structural variant or detected SNP or a sequence variant in a viral genome), one or more assembled consensus sequences and/or the a validation statistic for the one or more assembled consensus sequences. The report may be distributed to or the information may be reported to a recipient, for example a clinician, the subject, or a researcher.

### IV. Optical Systems

In some embodiments, provided herein is a total internal reflection fluorescence (TIRF) imaging system (e.g., a system for TIRF microscopy), and a method for using the TIRF imaging system for detecting and processing optical signals for nucleic acid (e.g., DNA or RNA) sequencing.

In some embodiments, provided herein is a cheap and simple TIRF imaging system for use in a user facing analytical equipment, e.g., for nucleic acid sequencing. Existing TIRF platforms either use objective style TIRF optics (which is expensive, and typically requires immersion oil between the lens and the substrate such as a cover slip, e.g., a cover glass) or prism style TIRF optics (which usually require low autofluorescence fused silica prisms, and immersion/optical matching oil between the substrate and the prism). In some embodiments, a prism-style TIRF platform is attractive because cheaper low numerical aperture (NA) objective lens can be used. Numerical Aperture (also termed Object-Side Aperture) is a value for microscope objectives and condensers: NA=n×sin(μ) or n×sin(α), where n represents the refractive index of the medium between the objective front lens and the specimen, and μ or α is the one-half angular aperture of the objective. The numerical aperture of a microscope objective is a measure of its ability to gather light and resolve fine specimen detail at a fixed object distance.

However, in some embodiments, in a user facing device dealing with oil on the prism is messy and inconvenient. To address these issues, in some embodiments, the prism is embedded in the substrate. In some embodiments, the prism is used as the substrate, making this component disposable, but where fused silica prisms are used this is cost prohibitive. In some embodiments, the fused silica prism can be replaced with a low auto-florescence plastic, for example ZEONEX 5000*. A plastic may be chosen to show minimal auto-florescence for a give excitation wavelength. In some embodiments, the prism comprises one or more optical quality plastic materials with a low autofluorescence, for use in detection by fluorescence and laser induced fluorescence techniques. For example, PDMS shows a comparatively low auto-florescence compared to other common plastics and can be used as a prism in a TIRF imaging system disclosed herein. In some embodiments, the prism comprises one or more commercially available plastic chip materials, such as PMMA, COC, PC, and/or PDMS. See, e.g., Piruska et al., “The autofluorescence of plastic materials and chips measured under laser irradiation,” Lab Chip, 2005, 5, 1348-1354, incorporated herein by reference in its entirety for all purposes.

In some embodiments, a plastic prism may form part of a disposable flowcell or flowcell/reagent cartridge. In some embodiments, the prism surface itself may be used as a substrate for the attachment of analytes to be imaged. In some embodiments, the prism may be bonded to a substrate.

In some embodiments, in order to reduce the effect of auto-florescence, an excitation filter may be used below and/or above the substrate. In some embodiments, the excitation filter is selected such that it passes the excitation wavelength and blocks auto-florescence. In some embodiments, the excitation filter blocks at least about 10%, at least about 20%, at least about 30%, at least about 40%, at least about 50%, at least about 60%, at least about 70%, at least about 80%, at least about 90%, or about 100% auto-florescence. Alternatively or in addition to the excitation filter, an additive may be added to the prism plastic to act as a filter. In some embodiments, a system disclosed herein can be used in combination with an emission filter.

In some embodiments, a lightguide style TIRF may be used. In some embodiments, a lightguide is integrated into a flowcell.

Systems as described above may be of utility in diagnostic or life science equipment and elsewhere. In some embodiments, an optical system disclosed herein may form part of a DNA or RNA sequencing instrument. In some embodiments, an optical system disclosed herein may be incorporated into a DNA or RNA sequencing system where a low cost optical approach to single molecule imaging is desirable. In some embodiments, a TIRF prism may be incorporated into a disposable cartridge or flowcell.

### V. Compositions, Kits, and Applications

Also provided herein are compositions and kits comprising one or more of the primers, nucleic acid molecules, substrates, nucleotides including detectably labeled nucleotides, polymerases, and reagents for performing the methods provided herein, for example reagents required for one or more steps comprising hybridization, ligation, amplification, detection, sequencing, and/or sample preparation as described herein, for example, in Section III.

The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for sample processing, such as nucleic acid extraction, isolation, and/or purification, e.g., RNA extraction, isolation, and/or purification. In some embodiments, the kits contain reagents, such as enzymes and buffers for ligation and/or amplification, such as ligases and/or polymerases. In some embodiments, the kits contain reagents, such as enzymes and buffers for primer extension and/or nucleic acid sequencing, such as polymerases and/or transcriptases. In some aspects, the kit can also comprise any of the reagents described herein, e.g., buffer components for tuning the rate of nucleotide incorporation and/or for tuning the rate of signal deactivation (e.g., by photobleaching). In some embodiments, the kits contain reagents for signal detection during sequencing, such as detectable labels and detectably labeled molecules. In some embodiments, the kits optionally contain other components, for example nucleic acid primers, enzymes and reagents, buffers, nucleotides, modified nucleotides, and reagents for additional assays.

In some aspects, the provided embodiments can be applied in analyzing nucleic acid sequences, such as DNA and/or RNA sequencing, for example single molecule real-time DNA and/or RNA sequencing. In some aspects, the embodiments can be applied in an imaging or detection method for multiplexed nucleic acid analysis. In some aspects, the provided embodiments can be used to identify or detect regions of interest in target nucleic acids, such as viral DNA or RNA. In some embodiments, the region of interest comprises one or more nucleotide residues, such as a single-nucleotide polymorphism (SNP), a single-nucleotide variant (SNV), substitutions such as a single-nucleotide substitution, mutations such as a point mutation, insertions such as a single-nucleotide insertion, deletions such as a single-nucleotide deletion, translocations, inversions, duplications, and/or other sequences of interest.

In some aspects, the embodiments can be applied in investigative and/or diagnostic applications, for example, for characterization or assessment of a sample from a subject. Applications of the provided method can comprise biomedical research and clinical diagnostics. For example, in biomedical research, applications comprise, but are not limited to, genetic and genomic analysis for biological investigation or drug screening. In clinical diagnostics, applications comprise, but are not limited to, detecting gene markers such as disease, immune responses, bacterial or viral DNA/RNA for patient samples, loss of genetic heterozygosity, the presence of gene alleles indicative of a predisposition towards disease or good health, likelihood of responsiveness to therapy, or in personalized medicine or ancestry.

### VI. Terminology

Specific terminology is used throughout this disclosure to explain various aspects of the apparatus, systems, methods, and compositions that are described.

Having described some illustrative embodiments of the present disclosure, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the present disclosure. In particular, although many of the examples presented herein involve specific combinations of method acts or system elements, it should be understood that those acts and those elements may be combined in other ways to accomplish the same objectives.

As used herein, the singular forms “a,” “an,” and “the” include plural referents unless the context clearly dictates otherwise. For example, “a” or “an” means “at least one” or “one or more.”

The term “about” as used herein refers to the usual error range for the respective value readily known to the skilled person in this technical field. Reference to “about” a value or parameter herein includes (and describes) embodiments that are directed to that value or parameter per se.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

The terms “nucleic acid” and “nucleotide” are intended to be consistent with their use in the art and to include naturally-occurring species or functional analogs thereof. Particularly useful functional analogs of nucleic acids are capable of hybridizing to a nucleic acid in a sequence-specific fashion (e.g., capable of hybridizing to two nucleic acids such that ligation can occur between the two hybridized nucleic acids) or are capable of being used as a template for replication of a particular nucleotide sequence. Naturally-occurring nucleic acids generally have a backbone containing phosphodiester bonds. An analog structure can have an alternate backbone linkage including any of a variety of those known in the art. Naturally-occurring nucleic acids generally have a deoxyribose sugar (e.g., found in deoxyribonucleic acid (DNA)) or a ribose sugar (e.g. found in ribonucleic acid (RNA)).

A nucleic acid can contain nucleotides having any of a variety of analogs of these sugar moieties that are known in the art. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G). Useful non-native bases that can be included in a nucleic acid or nucleotide are known in the art.

A “probe” or a “target,” when used in reference to a nucleic acid or sequence of a nucleic acids, is intended as a semantic identifier for the nucleic acid or sequence in the context of a method or composition, and does not limit the structure or function of the nucleic acid or sequence beyond what is expressly indicated.

The terms “oligonucleotide” and “polynucleotide” are used interchangeably to refer to a single-stranded multimer of nucleotides from about 2 to about 500 nucleotides in length. Oligonucleotides can be synthetic, made enzymatically (e.g., via polymerization), or using a “split-pool” method. Oligonucleotides can include ribonucleotide monomers (e.g., can be oligoribonucleotides) and/or deoxyribonucleotide monomers (e.g., oligodeoxyribonucleotides). In some examples, oligonucleotides can include a combination of both deoxyribonucleotide monomers and ribonucleotide monomers in the oligonucleotide (e.g., random or ordered combination of deoxyribonucleotide monomers and ribonucleotide monomers). An oligonucleotide can be 4 to 10, 10 to 20, 21 to 30, 31 to 40, 41 to 50, 51 to 60, 61 to 70, 71 to 80, 80 to 100, 100 to 150, 150 to 200, 200 to 250, 250 to 300, 300 to 350, 350 to 400, or 400-500 nucleotides in length, for example. Oligonucleotides can include one or more functional moieties that are attached (e.g., covalently or non-covalently) to the multimer structure. For example, an oligonucleotide can include one or more detectable labels (e.g., a radioisotope or fluorophore).

The terms “detectable label,” “optical label,” and “label” are used interchangeably herein to refer to a directly or indirectly detectable moiety that is coupled to or may be coupled to another moiety, for example, a nucleotide or nucleotide analog. The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. The label can emit a signal or alter a signal delivered to the label so that the presence or absence of the label can be detected. In some cases, coupling may be via a linker, which may be cleavable, such as photo-cleavable (e.g., cleavable under ultra-violet light), chemically-cleavable (e.g., via a reducing agent, such as dithiothreitol (DTT), tris(2-carboxyethyl)phosphine (TCEP)) or enzymatically cleavable (e.g., via an esterase, lipase, peptidase, or protease).

In some embodiments, a detectable label is or includes a fluorophore. Exemplary fluorophores include, but are not limited to, fluorescent nanocrystals; quantum dots; d-Rhodamine acceptor dyes including dichloro[R110], dichloro[R6G], dichloro[TAMRA], dichloro[ROX] or the like; fluorescein donor dye including fluorescein, 6-FAM, or the like; Cyanine dyes such as Cy3B; Alexa dyes, SETA dyes, Atto dyes such as atto 647N which forms a FRET pair with Cy3B and the like. Fluorophores include, but are not limited to, MDCC (7-diethylamino-3-[([(2-maleimidyl)ethyl]amino)carbonyl]coumarin), TET, HEX, Cy3, TMR, ROX, Texas Red, Cy5, LC red 705 and LC red 640.

In some embodiments, a detectable label is or includes a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. These protein moieties can catalyze chemiluminescent reactions given the appropriate substrates (e.g., an oxidizing reagent plus a chemiluminescent compound. A number of compound families are known to provide chemiluminescence under a variety of conditions. Non-limiting examples of chemiluminescent compound families include 2,3-dihydro-1,4-phthalazinedione luminol, 5-amino-6,7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and -methoxy substituents, oxalates such as oxalyl active esters, p-nitrophenyl, N-alkyl acridinum esters, luciferins, lucigenins, or acridinium esters. In some embodiments, a detectable label is or includes a metal-based or mass-based label.

The terms “hybridizing,” “hybridize,” “annealing,” and “anneal” are used interchangeably in this disclosure, and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

A “primer” is a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence.

A “nucleic acid extension” generally involves incorporation of one or more nucleic acids (e.g., A, G, C, T, U, nucleotide analogs, or derivatives thereof) into a molecule (such as, but not limited to, a nucleic acid sequence) in a template-dependent manner, such that consecutive nucleic acids are incorporated by an enzyme (such as a polymerase or reverse transcriptase), thereby generating a newly synthesized nucleic acid molecule. Enzymatic extension can be performed by an enzyme including, but not limited to, a polymerase and/or a reverse transcriptase. For example, a primer that hybridizes to a complementary nucleic acid sequence can be used to synthesize a new nucleic acid molecule by using the complementary nucleic acid sequence as a template for nucleic acid synthesis. Similarly, a 3′ polyadenylated tail of an mRNA transcript that hybridizes to a poly (dT) sequence can be used as a template for single-strand synthesis of a corresponding cDNA molecule. Furthermore, a poly (dT) sequence may be used as a sequencing primer for sequencing RNA molecules comprising poly(A) tails.

A “non-terminating nucleotide” or “incorporating nucleotide” can include a nucleic acid moiety that can be attached to a 3′ end of a polynucleotide using a polymerase or transcriptase, and that can have another non-terminating nucleic acid attached to it using a polymerase or transcriptase without the need to remove a protecting group or reversible terminator from the nucleotide. Naturally occurring nucleic acids are a type of non-terminating nucleic acid. Non-terminating nucleic acids may be labeled or unlabeled.

A “PCR amplification” refers to the use of a polymerase chain reaction (PCR) to generate copies of genetic material, including DNA and RNA sequences. Suitable reagents and conditions for implementing PCR are described, for example, in U.S. Pat. Nos. 4,683,202, 4,683,195, 4,800,159, 4,965,188, and 5,512,462, the entire contents of each of which are incorporated herein by reference. In a typical PCR amplification, the reaction mixture includes the genetic material to be amplified, an enzyme, one or more primers that are employed in a primer extension reaction, and reagents for the reaction. The oligonucleotide primers are of sufficient length to provide for hybridization to complementary genetic material under annealing conditions. The length of the primers generally depends on the length of the amplification domains, but will typically be at least 4 bases, at least 5 bases, at least 6 bases, at least 8 bases, at least 9 bases, at least 10 base pairs (bp), at least 11 bp, at least 12 bp, at least 13 bp, at least 14 bp, at least 15 bp, at least 16 bp, at least 17 bp, at least 18 bp, at least 19 bp, at least 20 bp, at least 25 bp, at least 30 bp, at least 35 bp, and can be as long as 40 bp or longer, where the length of the primers will generally range from 18 to 50 bp. The genetic material can be contacted with a single primer or a set of two primers (forward and reverse primers), depending upon whether primer extension, linear or exponential amplification of the genetic material is desired.

In some embodiments, the PCR amplification process uses a DNA polymerase enzyme. The DNA polymerase activity can be provided by one or more distinct DNA polymerase enzymes. In some embodiments, the DNA polymerase enzyme is from a bacterium, e.g., the DNA polymerase enzyme is a bacterial DNA polymerase enzyme. For instance, the DNA polymerase can be from a bacterium of the genus Escherichia, Bacillus, Thermophilus, or Pyrococcus.

In some embodiments, PCR amplification can include reactions such as, but not limited to, a strand-displacement amplification reaction, a rolling circle amplification reaction (e.g., the multiple repeats can be cleaved from the rolling circle amplification product), a ligase chain reaction, a transcription-mediated amplification reaction, an isothermal amplification reaction, and/or a loop-mediated amplification reaction.

In some embodiments, PCR amplification uses a single primer that is complementary to the 3′ tag of target DNA fragments. In some embodiments, PCR amplification uses a first and a second primer, where at least a 3′ end portion of the first primer is complementary to at least a portion of the 3′ tag of the target nucleic acid fragments, and where at least a 3′ end portion of the second primer exhibits the sequence of at least a portion of the 5′ tag of the target nucleic acid fragments. In some embodiments, a 5′ end portion of the first primer is non-complementary to the 3′ tag of the target nucleic acid fragments, and a 5′ end portion of the second primer does not exhibit the sequence of at least a portion of the 5′ tag of the target nucleic acid fragments. In some embodiments, the first primer includes a first universal sequence and/or the second primer includes a second universal sequence.

The term “DNA polymerase” includes not only naturally-occurring enzymes but also all modified derivatives thereof, including also derivatives of naturally-occurring DNA polymerase enzymes. For instance, in some embodiments, the DNA polymerase can have been modified to remove 5′-3′ exonuclease activity. Sequence-modified derivatives or mutants of DNA polymerase enzymes that can be used include, but are not limited to, mutants that retain at least some of the functional, e.g. DNA polymerase activity of the wild-type sequence. Mutations can affect the activity profile of the enzymes, e.g. enhance or reduce the rate of polymerization, under different reaction conditions, e.g. temperature, template concentration, primer concentration, etc. Mutations or sequence-modifications can also affect the exonuclease activity and/or thermostability of the enzyme.

Suitable examples of DNA polymerases that can be used include, but are not limited to: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion®DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes.

In some embodiments, genetic material is amplified by reverse transcription polymerase chain reaction (RT-PCR). The desired reverse transcriptase activity can be provided by one or more distinct reverse transcriptase enzymes, suitable examples of which include, but are not limited to: M-MLV, MuLV, AMV, HIV, ArrayScript™ MultiScribe™, ThermoScript™, and SuperScript® I, II, III, and IV enzymes. “Reverse transcriptase” includes not only naturally occurring enzymes, but all such modified derivatives thereof, including also derivatives of naturally-occurring reverse transcriptase enzymes.

In addition, reverse transcription can be performed using sequence-modified derivatives or mutants of M-MLV, MuLV, AMV, and HIV reverse transcriptase enzymes, including mutants that retain at least some of the functional, e.g. reverse transcriptase, activity of the wild-type sequence. The reverse transcriptase enzyme can be provided as part of a composition that includes other components, e.g. stabilizing components that enhance or improve the activity of the reverse transcriptase enzyme, such as RNase inhibitor(s), inhibitors of DNA-dependent DNA synthesis, e.g. actinomycin D. Many sequence-modified derivative or mutants of reverse transcriptase enzymes, e.g., M-MLV, and compositions including unmodified and modified enzymes are commercially available, e.g., ArrayScript™, MultiScribe™, ThermoScript™, and SuperScript® I, II, III, and IV enzymes.

Certain reverse transcriptase enzymes (e.g. Avian Myeloblastosis Virus (AMV) Reverse Transcriptase and Moloney Murine Leukemia Virus (M-MuLV, MMLV) Reverse Transcriptase) can synthesize a complementary DNA strand using both RNA (cDNA synthesis) and single-stranded DNA (ssDNA) as a template. Thus, in some embodiments, the reverse transcription reaction can use an enzyme (reverse transcriptase) that is capable of using both RNA and ssDNA as the template for an extension reaction, e.g., an AMV or MMLV reverse transcriptase.

### EXAMPLES

The following examples are included for illustrative purposes only and are not intended to limit the scope of the present disclosure.

### Example 1: Using Labels with Differing Intensities for Detecting Nucleotide Incorporation and/or Label Photobleaching Events During Sequencing-by-Synthesis

Clusters are grown using established methods, for example, on the Illumina Genome Analyzer 2, clusters are approximately 1 micron in diameter. Each cluster contains 500 to 1000 clonal copies. Super-resolution techniques can resolve individual labels at a resolution of 10 nm or less. A depletion ratio of 1:10 is used to sufficiently deactivate strands such that individual strands may be resolved. In one particular example, a ratio of 9 terminated nucleotides to 1 reversibly terminated nucleotide is used. As such 9 out of 10 strands are terminated and deactivated, leaving 50 to 100 active strands within a cluster covering a ˜1 micron diameter area, with an average distance between active strands of 88 nm. At this distance an optical super-resolution technique is able to resolve individual strands. For example, a super-resolution method using blinking labels (PAINT) or structured illumination methods is used.

Each of these strand is independently sequenced using sequencing-by-synthesis. Each strand is basecalled independently and shows no phasing artifacts. Once sequencing is complete (cyclic or otherwise), individual strand sequences are combined. For example, a simple consensus using multiple-alignment of all strand sequences is used. Alternatively positional information is incorporated, for example, if a subset of strands near the edge of the cluster show the same basecall that differs from those elsewhere in the cluster, this suggests a late strand error introduced during the cluster amplification process. Such errors are identified and removed. Using this process it is likely all but first cycle bridge amplification errors can be removed.

The present disclosure is not intended to be limited in scope to the particular disclosed embodiments, which are provided, for example, to illustrate various aspects of the disclosure. Various modifications to the compositions and methods described will become apparent from the description and teachings herein. Such variations may be practiced without departing from the true scope and spirit of the disclosure and are intended to fall within the scope of the present disclosure.

1. A method for nucleic acid sequencing, comprising:
a) contacting a cluster immobilized on a substrate with a primer and terminated nucleotide molecules which may but do not need to be detectably labeled,
wherein the cluster comprises nucleic acid molecules each comprising a common nucleic acid sequence to be sequenced, and
wherein at a first subset of the nucleic acid molecules in the cluster, a terminated nucleotide molecule is incorporated into the primer hybridized to each nucleic acid molecule in the first subset using the nucleic acid molecule as template, thereby deactivating the nucleic acid molecule by preventing phosphodiester bond formation of a nucleotide with the incorporated terminated nucleotide molecule, whereas a second subset of the nucleic acid molecules in the cluster are not deactivated;
b) contacting the cluster with a plurality of nucleotides comprising detectably labeled nucleotide molecules, wherein nucleotides are not incorporated at the deactivated nucleic acid molecules in the first subset, and a detectably labeled nucleotide molecule is incorporated at a non-deactivated nucleic acid molecule in the second subset using the non-deactivated nucleic acid molecule as template, optionally wherein the detectably labeled nucleotide molecule is incorporated into the primer or an extension product thereof hybridized to the non-deactivated nucleic acid molecule in the second subset using the non-deactivated nucleic acid molecule as template; and
c) detecting signals associated with the incorporation of detectably labeled nucleotide molecules at individual nucleic acid molecules in the cluster, thereby determining a sequence of the common nucleic acid sequence to be sequenced.

2. The method of claim 1, wherein a plurality of clusters are immobilized on the substrate, each comprising clonal copies of a common nucleic acid sequence to be sequenced.

3. The method of claim 1 or 2, wherein at least 100, at least 1,000, at least 10,000, at least 100,000, at least 1,000,000, or more than 1,000,000 clusters are immobilized on the substrate.

4. The method of claim 2 or 3, wherein at least 100, at least 1,000, at least 10,000, at least 100,000, at least 1,000,000, or more than 1,000,000 different common nucleic acid sequences to be sequenced are in the clusters immobilized on the substrate.

5. The method of any of claims 1-4, wherein the terminated nucleotide molecules comprise A nucleotides, T/U nucleotides, C nucleotides, and/or G nucleotides.

6. The method of any of claims 1-5, wherein the terminated nucleotide molecules contain only one, only two, only three, or all four of A nucleotides, T/U nucleotides, C nucleotides, and G nucleotides.

7. The method of any of claims 1-6, wherein in a), the cluster is contacted with a plurality of nucleotide molecules comprising the terminated nucleotide molecules and non-terminated nucleotide molecules.

8. The method of claim 7, wherein the non-terminated nucleotide molecules comprise A nucleotides, T/U nucleotides, C nucleotides, and/or G nucleotides.

9. The method of claim 7 or 8, wherein the non-terminated nucleotide molecules contain only one, only two, only three, or all four of A nucleotides, T/U nucleotides, C nucleotides, and G nucleotides.

10. The method of any of claims 7-9, wherein the plurality of nucleotide molecules comprise:
i) terminated A nucleotide molecules and non-terminated A nucleotide molecules;
ii) terminated T/U nucleotide molecules and non-terminated T/U nucleotide molecules;
iii) terminated C nucleotide molecules and non-terminated C nucleotide molecules; and/or
iv) terminated G nucleotide molecules and non-terminated G nucleotide molecules.

11. The method of any of claims 7-10, wherein the ratio of terminated nucleotide molecules to non-terminated nucleotide molecules in the plurality of nucleotide molecules is at least or about 1:10, at least or about 1:8, at least or about 1:6, at least or about 1:4, at least or about 1:2, at least or about 1:1, at least or about 2:1, at least or about 4:1, at least or about 6:1, at least or about 8:1, at least or about 10:1, at least or about 20:1, at least or about 50:1, at least or about 100:1, at least or about 200:1, or at least or about 500:1.

12. The method of any of claims 7-11, wherein:
i) the ratio of terminated A nucleotide molecules to non-terminated A nucleotide molecules is between about 1:4 and about 4:1;
ii) the ratio of terminated T/U nucleotide molecules to non-terminated T/U nucleotide molecules is between about 1:4 and about 4:1;
iii) the ratio of terminated C nucleotide molecules to non-terminated C nucleotide molecules is between about 1:4 and about 4:1; and/or
iv) the ratio of terminated G nucleotide molecules to non-terminated G nucleotide molecules is between about 1:4 and about 4:1.

13. The method of any of claims 7-12, wherein the cluster is contacted with:
i) terminated A nucleotide molecules and non-terminated A nucleotide molecules, ii) terminated T/U nucleotide molecules and non-terminated T/U nucleotide molecules,
iii) terminated C nucleotide molecules and non-terminated C nucleotide molecules, and
iv) terminated G nucleotide molecules and non-terminated G nucleotide molecules.

14. The method of claim 13, wherein the cluster is contacted with any two, any three, or all four of i), ii), iii), and iv) pre-mixed in a mixture.

15. The method of claim 13, wherein the cluster is contacted with i), ii), iii), and iv) sequentially in separate cycles.

16. The method of any of claims 1-15, wherein the terminated nucleotide molecules comprise irreversibly terminated nucleotide molecules.

17. The method of any of claims 1-16, wherein the terminated nucleotide molecules comprise ddNTP.

18. The method of any of claims 1-17, wherein the terminated nucleotide molecules comprise reversibly terminated nucleotide molecules.

19. The method of claim 18, which does not comprise removing a reversible terminating group to render the reversibly terminated nucleotide molecules capable of forming phosphodiester bonds after incorporation of the reversibly terminated nucleotide molecules.

20. The method of any of claims 1-19, wherein the ratio of the number of molecules in the first subset to that in the second subset is at least or about 1:10, at least or about 1:8, at least or about 1:6, at least or about 1:4, at least or about 1:2, at least or about 1:1, at least or about 2:1, at least or about 4:1, at least or about 6:1, at least or about 8:1, at least or about 10:1, at least or about 20:1, at least or about 50:1, at least or about 100:1, at least or about 200:1, or at least or about 500:1.

21. The method of any of claims 1-20, wherein the ratio of the number of molecules in the first subset to that in the second subset is between about 1:4 and about 4:1.

22. The method of any of claims 1-21, wherein the density of non-deactivated nucleic acid molecules in the cluster is one molecule per at least about 250 nm², one molecule per at least about 200 nm², one molecule per at least about 150 nm², one molecule per at least about 100 nm², one molecule per at least about 50 nm², or one molecule per at least about 20 nm², or any value in between the aforementioned values.

23. The method of any of claims 1-22, wherein the detectably labeled nucleotide molecules comprise the same detectable label.

24. The method of any of claims 1-23, wherein the detectably labeled nucleotide molecules comprise two, three, four, or more different detectable labels.

25. The method of any of claims 1-24, wherein among the detectably labeled nucleotide molecules, two or more nucleotides comprising the same base are labeled with different detectable labels, and/or two or more nucleotides comprising different bases are labeled with the same detectable label.

26. The method of any of claims 1-24, wherein among the detectably labeled nucleotide molecules, nucleotides comprising the same base are labeled with the same detectable label, and nucleotides comprising different bases are labeled with different detectable labels each corresponding to a different base, optionally wherein A, T/U, C, and G each corresponds to a fluorophore identifying the base from among the four bases.

27. The method of any of claims 1-26, wherein the primer hybridizes to the nucleic acid molecule at a sequence that is 3′ to the common nucleic acid sequence to be sequenced.

28. The method of any of claims 1-27, wherein the detectably labeled nucleotide molecules are incorporated using the common nucleic acid sequence to be sequenced as template, thereby determining the sequence of the common nucleic acid sequence.

29. The method of any of claims 1-28, wherein the non-deactivated nucleic acid molecules are sequenced using a single molecule real-time sequencing method.

30. The method of any of claims 1-29, wherein the substrate comprises a bead, a planar substrate, a solid surface, a flow cell, a semiconductor chip, a well (optionally a microwell), a pillar (optionally a micropillar), a chamber (optionally a microchamber), a channel (optionally a microchannel), a through hole, a nanopore, or any combination thereof.

31. The method of any of claims 1-30, wherein the nucleic acid molecules comprise DNA and/or RNA.

32. The method of any of claims 1-31, wherein in b), the plurality of nucleotides contacted with the cluster comprise nucleotide molecules that are non-terminated and nucleotide molecules that are reversibly terminated.

33. The method of claim 32, wherein the non-terminated nucleotide molecules comprise detectably labeled nucleotide molecules and/or non-detectably labeled nucleotide molecules.

34. The method of claim 32 or 33, wherein the reversibly terminated nucleotide molecules comprise detectably labeled nucleotide molecules and/or non-detectably labeled nucleotide molecules.

35. The method of any of claims 32-34, wherein the non-terminated nucleotide molecules are non-detectably labeled, and the reversibly terminated nucleotide molecules are detectably labeled.

36. The method of any of claims 32-34, wherein the non-terminated nucleotide molecules are detectably labeled, and the reversibly terminated nucleotide molecules are non-detectably labeled.

37. The method of any of claims 32-36, wherein the reversibly terminated nucleotide molecules are incorporated and terminate stochastically at nucleic acid molecules in the cluster, thereby increasing phasing among non-deactivated nucleic acid molecules compared to that among non-deactivated nucleic acid molecules contacted with only non-terminated nucleotide molecules or with only reversibly terminated nucleotide molecules for sequencing.

38. The method of any of claims 1-37, wherein in a), the cluster is contacted with a plurality of primers each comprising a sequence complementary to a different region in the common nucleic acid sequence to be sequenced.

39. The method of claim 38, wherein a terminated nucleotide molecule is incorporated into at least some of the plurality of primers hybridized in or adjacent to the common nucleic acid sequence to be sequenced.

40. The method of claim 38 or 39, comprising determining a sequence of the common nucleic acid sequence using at least some of the different primers hybridized to the non-deactivated nucleic acid molecules in the cluster.

41. The method of claim 40, wherein the sequences determined using the different primers are analyzed using multiple alignment.

42. The method of claim 40 or 41, wherein the sequences determined using the different primers are synthesized to form a synthetic long read sequence of at least or about 100, at least or about 200, at least or about 500, at least or about 1,000, at least or about 2,000, or at least or about 5,000 nucleotides in length.

43. The method of any of claims 1-42, wherein the signals associated with the incorporation of detectably labeled nucleotide molecules are detected using a total internal reflection fluorescence (TIRF) imaging system.

44. The method of claim 43, wherein the TIRF imaging system comprises a prism comprising a low auto-florescence plastic material, optionally the prism is used as at least a portion of the substrate.

45. The method of claim 43 or 44, wherein the TIRF imaging system comprises an excitation filter below and/or above the substrate.
